SEQUENCE LISTING (29 sequences)

SEQ ID NO: 1 - RNA, 23 nt, Homo sapiens
ucuuugguuaucuagcuguauga

SEQ ID NO: 2 - RNA, 23 nt, Homo sapiens
caaagugcucauagugcagguag

SEQ ID NO: 3 - RNA, 22 nt, Homo sapiens
cauugcacuugucucggucuga

SEQ ID NO: 4 - RNA, 23 nt, Homo sapiens
caaagugcuguucgugcagguag

SEQ ID NO: 5 - RNA, 21 nt, Homo sapiens
uaaagugcugacagugcagau

SEQ ID NO: 6 - RNA, 21 nt, Homo sapiens
agcuacaucuggcuacugggu

SEQ ID NO: 7 - RNA, 22 nt, Homo sapiens
aaaagcuggguugagagggcga

SEQ ID NO: 8 - RNA, 23 nt, Homo sapiens
cgcauccccuagggcauuggugu

SEQ ID NO: 9 - RNA, 22 nt, Homo sapiens
aauugcacgguauccaucugua

SEQ ID NO: 10 - RNA, 22 nt, Homo sapiens
ucguaccgugaguaauaaugcg

SEQ ID NO: 11 - RNA, 21 nt, Homo sapiens
ugagaugaagcacuguagcuc

SEQ ID NO: 12 - RNA, 23 nt, Homo sapiens
guccaguuuucccaggaaucccu

SEQ ID NO: 13 - RNA, 22 nt, Homo sapiens
acaguagucugcacauugguua

SEQ ID NO: 14 - RNA, 23 nt, Artificial Sequence
  miR-9-5p primer
ucauacagcuagauaaccaaaga

SEQ ID NO: 15 - RNA, 22 nt, Artificial Sequence
  miR-20b-5p primer
cuaccugcacuaugagcauuug

SEQ ID NO: 16 - RNA, 22 nt, Artificial Sequence
  miR-25-3p primer
ucagaccgagacaagugcaaug

SEQ ID NO: 17 - RNA, 23 nt, Artificial Sequence
  miR-93-5p primer
cuaccugcacgaacagcacuuug

SEQ ID NO: 18 - RNA, 21 nt, Artificial Sequence
  miR-106b-5p primer
aucugcacugucagcacuuua

SEQ ID NO: 19 - RNA, 21 nt, Artificial Sequence
  miR-222-3p primer
acccaguagccagauguagcu

SEQ ID NO: 20 - RNA, 22 nt, Artificial Sequence
  miR-320a primer
ucgcccucucaacccagcuuuu

SEQ ID NO: 21 - RNA, 23 nt, Artificial Sequence
  miR-324-5p primer
acaccaaugcccuaggggaugcg

SEQ ID NO: 22 - RNA, 22 nt, Artificial Sequence
  miR-363-3p primer
uacagauggauaccgugcaauu

SEQ ID NO: 23 - RNA, 22 nt, Artificial Sequence
  miR-126-3p primer
cgcauuauuacucacgguacga

SEQ ID NO: 24 - RNA, 21 nt, Artificial Sequence
  miR-143-3p primer
gagcuacagugcuucaucuca

SEQ ID NO: 25 - RNA, 23 nt, Artificial Sequence
  miR-145 5p primer
agggauuccugggaaaacuggac

SEQ ID NO: 26 - RNA, 22 nt, Artificial Sequence
  miR-100a-3p primer
uaaccaaugugcagacuacugu

SEQ ID NO: 27 - DNA, 23 bp, Artificial Sequence
  LNA probe
tcatacagctagataaccaaaga

SEQ ID NO: 28 - DNA, 21 bp, Artificial Sequence
  LNA probe
atctgcactgtcagcacttta

SEQ ID NO: 29 - DNA, 23 bp, Artificial Sequence
  LNA probe
gaacaggtagtctgaacactggg

(19) United States
(12) Patent Application Publication
(10) Pub. No.: US 2016/0208346 A1
(43) Pub. Date: Jul. 21, 2016

(54) METHOD AND COMPOSITION FOR DETECTION OF ONCOGENIC HPV

(71) Applicant: NOTRE DAME DU LAC, Notre Dame, IN (US)

(72) Inventors: Sharon Stack, Notre Dame, IN (US); Daniel L. Miller, Notre Dame, IN (US)

(21) Appl. No.: 14/912,898

(22) PCT Filed: Aug. 19, 2014
(86) PCT No.: PCT/US14/51692

§ 371 Date: Feb. 18, 2016

Related U.S. Application Data
(60) Provisional application No. 61/867,380, filed on Aug. 19, 2013.
(60) Provisional application No. 61/942,485, filed on Feb. 20, 2014.

(51) Int. Cl. C12Q 1/70

(52) U.S. Cl. CPC C12Q 1/708; C12Q 2600/178; C12Q 2600/158

(57) ABSTRACT

Probes for the detection of oral oncogenic human papillomavirus (HPV) are described. The probe includes a polynucleotide having at least 90% sequence identity to a polynucleotide complementary to a microRNA that has altered expression in response to oncogenic HPV infection. A method for detecting oncogenic HPV in a subject is also described. The method comprises the steps of (A) providing a sample from a subject; (B) measuring the expression level of a microRNA having altered expression in response to oncogenic HPV infection using a probe; and (C) determining that the subject is infected by an oncogenic HPV if the expression level is increased or decreased in comparison with a control.

### CROSS-REFERENCE TO RELATED APPLICATIONS

This application claims the benefit of U.S. Provisional Application No. 61/867,380, filed Aug. 19, 2013, and U.S. Provisional Application No. 61/942,485, filed Feb. 20, 2014, both of which are incorporated herein by reference.

### GOVERNMENT FUNDING

The present invention was made with government support under NIH-NIDCR F31DE021926-02 awarded by the National Institutes of Health-National Institute of Dental and Craniofacial Research (NIH-NIDCR). This invention was also made with government support under NIH-NCI RO1 CA085870 awarded by the National Institutes of Health-National Cancer Institute (NIH-NCI). The US government has certain rights in this invention.

### SEQUENCE LISTING

The instant application contains a Sequence Listing which has been submitted electronically in ASCII format and is hereby incorporated by reference in its entirety. Said ASCII copy, created on Aug. 17, 2014, is named UND-023707 Sequence_ST25 and is 5100 bytes in size.

### BACKGROUND

1. Field of the Invention

The present disclosure generally relates to methods of disease detection and prognosis and, more particularly, to methods for detecting oncogenic viruses, such as oral oncogenic HPV.

2. Description of Related Art

There are various techniques for detecting an infection of the head and neck. While the overall incidence of head and neck squamous cell carcinoma (HNSCC) has decreased since 1988, the incidence of tumors localized in the oropharynx has shown a striking 225% increase. With regards to head and neck cancer detection, current methods identify thyroid cancer, head and neck squamous cell carcinoma, or the relationship between two diseases in the head and neck, to name a few. See Miller et al., Biochem J. 443, 339-353 (2012). However, the detection of these diseases does not address the rising number of HPV-related cancers, particularly in the mouth.

Recently, the percentage of HPV-related diseases has been increasing. The ability to distinguish oncogenic human papillomavirus (HPV) versus non-oncogenic HPV is very important regarding the consequences to a patient. The presence of oncogenic HPV versus non-oncogenic HPV determines the further treatment of the patient. The current techniques to distinguish HPV-related cancer require two different sets of tests: one to determine HPV+ or HPV−, and an additional test is required to determine if the sample is oncogenic or not oncongenic. None of these techniques alone were particularly well suited to provide a sensitive, specific, and non-invasive way of differentiating oncogenic viruses from non-oncogenic infections, wherein the percentage of accuracy is relatively high. The optimal method to detect oral oncogenic HPV remains unaddressed by current techniques.

With regards to HPV detection, current methods provide cervical cancer diagnostic kits and therapies. However, these methods, among other things, do not address the population of those infected with oncogenic HPV, which can have substantially different infections. Furthermore, these methods require examination of samples that are taken invasively, by methods such as obtaining a blood sample or pap smear. Additionally, these methods typically involve the analysis of molecules like proteins or DNA, which have high false-positive detection rates.

What is not available is a test for distinguishing oncogenic viruses from non-oncogenic viruses that is more specific than proteins or DNA, using a sample that is obtained in a non-invasive way from patients. Such a test would provide comfort to the patient and a higher accuracy of detection.

Currently, clinicians use microRNAs to detect stomach cancers, pancreatic cancers, and cervical cancer. These methods cannot be made analogous to diseases of the head and neck, and certainly cannot address an HPV-related cancer in the mouth because of the genotypic differences in these variant diseases. None of these detection techniques provide a sensitive test for a non-invasively obtained sample. Accordingly, there is therefore a need for methods to more sensitively detect oncogenic HPV.

### SUMMARY OF THE INVENTION

High-risk human papillomavirus (HPV) is a causative agent for an increasing subset of oropharyngeal squamous cell carcinomas (OPSCC) and current evidence supports these tumors as having identifiable risk factors and improved response to therapy. However the biochemical and molecular alterations underlying the pathobiology of HPV-associated OPSCC (designated HPV+OPSCC) remain unclear. Herein we profile microRNA (miRNA) expression patterns in HPV+OPSCC to provide a more detailed understanding of pathological molecular events and to identify biomarkers that may have applicability for early diagnosis, improved staging, and prognostic stratification. Differentially expressed miRNAs were identified in RNA isolated from an initial clinical cohort of HPV+/−OPSCC tumors by qPCR-based miRNA profiling. This oncogenic miRNA panel was validated using miRNAseq and clinical data from The Cancer Genome Atlas (TCGA) and miRNA-in situ hybridization (miR-ISH). The HPV-associated oncogenic miRNA panel has potential utility in diagnosis and disease stratification as well as in mechanistic elucidation of molecular factors that contribute to OPSCC development, progression and differential response to therapy.

The shortcomings of the related art are overcome and additional advantages are provided through the provision of probes for the detection of oncogenic HPV. The probes are polynucleotide complementary to a microRNA having altered expression in response to an oncogenic HPV infection, and include probes having 90% or more sequence identity to such probes.

Additional shortcomings of the related art are overcome and additional advantages are provided through a method for detection of oncogenic HPV comprising providing a sample of oral rinse from a subject, measuring the expression level of microRNA having an altered level of expression in response to oncogenic HPV infection using a complementary probe, wherein the probe has at least 90% sequence identity to a directly complementary probe, and determining the presence of oncogenic HPV.

Additional features and advantages are realized through the techniques of the invention described herein. Other embodiments and aspects are described in detail herein and are considered a part of the claimed technology. For a better understanding of the advantages and features of the technology, refer to the description and the drawings.

### BRIEF DESCRIPTION OF THE FIGURES

The present invention may be more readily understood by reference to the following figures, wherein:

FIG. 1 provides a graph illustrating an example of small RNA expression across 13 samples. Micro- and Small nucleolar RNAs were used as positive controls to assess the quality of RNA extracted from Formalin-Fixed, Paraffin-Embedded (FFPE) tissues blocks that were obtained between the years 2006-2011. Because these universally expressed ncRNA were stable across samples, it was concluded that miRNA is relatively stable and protected in paraffin;

FIG. 2 provides a bar graph illustrating the p-values from miR-320a, miR-222-3p, and miR-93-5p based on the data shown in Table 2;

FIG. 3 provides a bar graph illustrating the p-values from miR-451a, miR-199a-3p//145-5p, miR-143-3p, miR-126-5p, and miR-126-3p based on the data shown in Table 3;

FIG. 4 provides a bar graph illustrating that miRs differentially expressed in 2 HPV+vs 2HPV− cell lines;

FIG. 5 illustrates levels of miR-145 repressed in HPV-31-positive organotypic rafts. (A) is an example of qPCR analysis of miR-145 levels in 13-day-old organotypic raft cultures of matched normal keratinocytes (viv) and HPV-positive cells that stably maintain episomes (viv-31gen). The data are represented as fold changes with respect to miR-145 levels. (B and C) are examples of luciferase reporter assays measuring responsiveness to miR-145 of sequences in HPV-31 E1 and E2. (D) is an example of mutation of miR-145 seed and are averages with standard errors from three independent experiments. (E) is an example of a schematic representation of HPV− 31 genome with miR-145 target sequences indicated;

FIG. 6 illustrates an example of levels of miR-145 decrease upon differentiation of HPV-31-positive cells. (A) is an example of E7 protein mediated repression of miR-145. The data are from qPCR and are normalized to U6 levels and are represented as fold difference from miR-145 levels in normal keratinocytes. (B) is an example of qPCR analysis of miR-145 levels in normal keratinocytes induced to differentiate in high calcium media. The data are normalized to U6 levels and are represented as fold change relative to miR-145 levels in undifferentiated cultures. (C) is an example of qPCR analysis of miR-145 levels in cells stably maintaining HPV-31 episome monolayer cultures upon differentiation in high calcium. The data are normalized to U6 levels and are represented as fold change from levels seen in undifferentiated cells;

FIG. 7 illustrates an example of high-level expression of miR-145 from heterologous expression vectors blocks HPV genome amplification, late gene expression, and induction of KLF-4. (A) is an example of a southern blot analysis of supercoiled episomal viral DNA levels upon differentiation of CIN-612 cells with forced expression of miR-145. Averages from three independent experiments with standard errors are shown in the bar graph. UD, undifferentiated. (B) is an example of a southern blot analysis of supercoiled episomal viral DNA levels following differentiation of CIN-612 cells expressing high levels of miR-146a (CIN-612 miR-146a cells), vector control, and mock-transfected cells. Quantification of the band intensities is shown in the bar graph. (C) is an example of a northern blot analysis of early and late viral transcripts during differentiation of CIN-612 miR-145 cells and mock-infected control cells. Arrows indicate early transcripts encoding E6*, E7, E1, E2, E5, E6*, E7, and E1Ê4, as well as late transcripts encoding E1Ê4 and E5. Results from three independent experiments with standard errors are shown. (D) is an example of a western blot analysis showing levels of KLF-4 and Oct-4 in cell extracts from raft cultures. A nonspecific band (at 60 kDa) was detected in CIN-612 cultures. The individual bands were quantified and normalized are represented as fold differences under each band.

FIG. 8 illustrates a scatter plot and graphical analysis of an example of a microarray analysis of microRNA expression showing HPV ‘over-expressed’ microRNA data from cell lines obtained using an exiqon microarray;

FIG. 9 illustrates a graphical example of a microarray analysis of relative microRNA expression through fold change showing HPV ‘over-expressed’ microRNA data from cell lines obtained using an exiqon microarray;

FIG. 10 illustrates an example of analysis on raw patient data from FFPE samples according to each patient for miR-320a, miR-93, and miR-222-3p;

FIG. 11 illustrates an example of analysis on raw patient data from FFPE samples according to each patient for miR-106a, miR-15a, and miR-141-;

FIG. 12 illustrates an example of analysis on raw patient data from FFPE samples according to each patient for miR-200c, miR-335, and miR-26b;

FIG. 13 illustrates an example of analysis on raw patient data from FFPE samples according to each patient for miR-33a;

FIG. 14 illustrates an example of analysis on raw patient data from FFPE samples according to each patient for miR-26b;

FIG. 15 illustrates an example of analysis on raw patient data from FFPE samples according to each patient for miR-34a, miR-145-5p, miR-143-3p and miR-451; and

FIG. 16 illustrates an example of analysis on raw patient data from FFPE samples according to each patient for miR-199a-3p//199b-3p, miR-126-3p, miR-199b-5p, and miR-126-5p.

FIG. 17 shows the HPV prevalence in FFPE cases. (A) Clinical history for patients diagnosed between 2006-2011 was reviewed, archived FFPE tissue blocks were assessed for available tissue, and available H&E slides reviewed. Following an IRB approved protocol, tissues were sectioned, stained for p16 protein expression and scored as positive or negative as described. Results show that 58% of cases are HPV+. (B) The average ages in the p16+ and p16− cohorts under study were 56.49 and 61.00 years of age, respectively.

FIG. 18 shows the profiling of miRNA expression on FFPE samples by q-rt-PCR. (A) Tumors from 23 patients, including fifteen p16+ and eight p16− samples were profiled as described. Panel shows a unsupervised hierarchical clustering heat map of normalized data (prior to non-specific filtering or testing) representing 511 miRNAs. The dendogram at the top of the heat map illustrates which patient samples have the most similar miRNA profile, while the dendrogram on the left y-axis illustrates which miRNAs have similar profiles across patients. Items which are most similar are linked sooner to each other than items which are less similar. The panel at the bottom provides clinical information for each sample, with a black square marking the presence of the indicated variable (gray indicates missing data); green (8/10 HPV+) and salmon (6/13 HPV−) shading indicate how the samples cluster into two groups. (B) Significantly up- or down-regulated miRNAs (p<0.01). (C) Unsupervised hierarchical clustering heatmap based on 43 selected miRNAs. The dendogram (top) is broken into 6 distinct groups with the majority of the samples falling into 3 groups: mostly smokers regardless of HPV status; all HPV+ non-smokers, and mixed.

FIG. 19 shows the analysis of TCGA Cohort 1 miRNAseq data. (A) Patients comprising TCGA Cohort 1 were identified as described. Graph shows comparison of the 7 differentially expressed miRNAs identified by PCR profiling of microdissected FFPE (fold changes in log 2 scale for symmetry) versus results obtained from analysis of TCGA Cohort 1 miRNA-seq data. A strong concordance between the datasets was obtained based on Spearmans rank correlation (Rho=0.85, p=0.02) and Pearson's product-moment correlation (r=0.83, p=0.02; 95% CI 0.21-0.97). A best-fit line indicates this relative concordance, while a 45-degree reference line (dotted) indicates that there is not perfect absolute agreement between the data sets and assay technique. (B) Mean normalized miRNA read counts showing miR-199a as a validated HPV-associated miRNA (p=9.8E-03). (C) miR-106b is expressed from the same primary polycistronic transcript as miR-93 (FIG. 2) and shares the same functionally relevant nucleotide (seed) sequence. Mean normalized miRNA read count shows upregulation of this paralog in HPV+ tumors (p=1.6E-04). (D) Mean normalized read counts showing miR-9 is significantly upregulated in HPV+(p=5.4E-04). This miRNA was upregulated, but not significantly in FFPE qPCR experiments (FIG. 2), and has been identified by two independent published studies as an HPV-associated miRNA in OPSCC [21, 27]. All bars indicate 95% confidence limits, not standard error of the mean. (E) Heatmap of normalized expression data with color scale representing greater or lesser levels of relative expression across TCGA HNSCC samples (columns) and miRs (rows). Unsupervised hierarchical clustering of both the samples and the miRs was carried out using Euclidean distance and the average linkage methods, and the resulting dendrograms are shown in the margins, where ‘+’ or ‘−’ indicates HPV status. Items that are most similar cluster lower in the dendrogram. There appear to be 2 distinct clusters of samples, one entirely HPV+ and the other mostly HPV−, as well as 4 distinct clusters of miRNAs. As an example reflecting the color scale, the bottom rows show miRNA expressed across all samples at levels between 15,000-320,000 read per million miRNA mapped.

FIG. 20 shows the analysis of TCGA Cohort 2 miRNAseq data. (A) Patients comprising TCGA Cohort 2 were identified as described. Graph shows comparison of the 7 differentially expressed miRNAs identified by PCR profiling of microdissected FFPE (fold changes in log 2 scale) versus results obtained from analysis of TCGA Cohort 2 miRNA-seq data. A strong concordance between datasets was obtained based on Spearmans rank correlation (Rho=0.75, p=0.06) and Pearson's product-moment correlation (r=0.78, p=0.03; 95% CI 0.07-0.96). A best-fit line indicates this relative concordance, while a 45-degree reference line (dotted) indicates that there is not perfect absolute agreement between the data sets and assay technique. (B) Mean normalized miRNA read counts showing miR-106b as a validated HPVassociated miRNA (p=5.4E-04). (C) Mean normalized miRNA read counts showing miR-9 as a validated HPV-associated miRNA (p=5.8E-04). All bars indicate 95% confidence limits, not standard error of the mean.

FIG. 21 shows the In-situ hybridization analysis of miR-9 expression in HNSCC. (A-D) Low power images depicting ISH patterns for miR-9 in four OPSCC tissue cores. The ISH signal is represented by a deep shade. The counter stain for miRNA-probed tissues sections was nuclear fast red; therefore, darker coloration represents ISH signal and lighter is the counterstain. (A,C) HPV+ tumors, (B,D) HPVtumors. HPV+ tumors show strong ISH signal while HPV− tumors have weak or absent signal. Magnification 100×. (E-H) High power images depicting ISH patterns for miR-9 in four OPSCC tissue cores. Staining in HPV+ cores is punctate (E) or diffuse (G) while HPV− tumors have weak or absent signal. Magnification 400×. (I) Odds ratios and mosaic plots of high tumor miR-9 occurring in the setting of p16+ disease. The odds that high-tumor miR-9 expression occurs in the setting of p16+ disease are more than 3 times greater (OR=3.38; p<0.001; 95% CI 1.84-6.26) than the odds of low-tumor miR-9 expression; similarly, the odds of diffuse miR-9 ISH are nearly four times greater (OR=3.87; p<0.001; 95% CI 2.10-7.20) than the odds of non-diffuse miR-9 ISH.(J) The odds ratios increase when the outcome is HPV mRNA ISH. The model prediction based on HPV mRNA ISH is associated has sensitivity=0.62 and specificity=0.875.

### DETAILED DESCRIPTION OF THE INVENTION

The present disclosure provides a method and composition that utilizes host-specific macromolecules, such as microRNAs, to detect the presence of oncogenic infections that previously were unable to be distinguished so specifically, without a high false-positive rate.

The following description of example methods and composition is not intended to limit the scope of the description to the precise form or form detailed herein. Instead the following description is intended to be illustrative so that others may follow its teachings.

As used herein, a subject can be a vertebrate, more specifically a mammal (e.g., a human, horse, cat, dog, cow, pig, sheep, goat, mouse, rabbit, rat, and guinea pig), birds, reptiles, amphibians, fish, and any other animal. The term does not denote a particular age. Thus, adult, juvenile, and newborn subjects are intended to be covered. As used herein, patient or subject may be used interchangeably and can refer to a subject afflicted with a disease or disorder (e.g. oncogenic HPV). The term patient or subject includes human and veterinary subjects.

The term “control” refers to an experiment or observation used to minimize the effects of variables. Through the comparison of a control measurement and a measurement, reliability can be increased. In one embodiment, a dataset may be obtained from samples from a group of subjects known to have a particular oncogenic infection. The expression data of the biomarkers in the dataset can be used to create a control value that is used in testing samples from new subjects. In such an embodiment, the control is a predetermined value for each biomarker or set of biomarkers obtained from subjects with oncogenic infection.

The term “polynucleotide” as used herein refers to a nucleic acid sequence including DNA, RNA, and microRNA and can refer to markers which are either double stranded or single stranded. Polynucleotide can also refer to synthetic variants with alternative sugars like the LNA.

The term “complementary” as used herein refers to nucleotide sequences that complement the polynucleotides' reverse sequence. Complementarity is the base principle of DNA replication and transcription as it is a property shared between two DNA or RNA sequences, such that when they are aligned antiparallel to each other, the nucleotide bases at each position in the sequences will be complementary. Complementarity is achieved by distinct interactions between nucleobases: adenine, thymine (uracil in RNA), guanine and cytosine. Adenine (A) and guanine (G) are purines, while thymine (T), cytosine (C) and uracil (U) are pyrimidines. Purines are larger than pyrimidines. Both types of molecules complement each other and can only base pair with the opposing type of nucleobase. In nucleic acid, nucleobases are held together by hydrogen bonding, which only works efficiently between adenine and thymine and between guanine and cytosine. The base complement A=T shares two hydrogen bonds, while the base pair GC has three hydrogen bonds.

The degree of complementarity between two nucleic acid strands may vary, from complete complementarity (each nucleotide is across from its opposite) to no complementary (each nucleotide is not across from its opposite) and determines the stability of the sequences to be together. Lesser degrees of complementarity are referred to herein by percentages of sequence identity as compared with a sequence having 100% complementarity. Embodiments of the invention include sequences having at least about 70% to at least about 100% sequence identify to a complementary sequence. For example, probes can have sequences having at least 70%, 75%, 80%, 85%, 90%, 95%, or 100% sequence identify with a complementary probe. In another example, the sequence identity can be at least about 80% to at least about 95% that of a complementary sequence. In a preferred embodiment, the probe can have at least about 87%, 88%, 89%, 90%, 91%, or 92% sequence identity to a complementary probe. In another preferred embodiment, the probe can have at least 90% sequence identity to a complementary probe.

The term “microRNAs” as used herein refers to a class of small RNAs typically between 15 and 30 nucleotides long. microRNAs can refer to a class of small RNAs that play a role in gene regulation. In a preferred embodiment, a microRNA refers to a human, small RNA of 20, 21, 22, 23, 24, 25, or 26 nucleotides long.

The term “expression” as used herein refers to the presence of biomarkers, such as the presence of microRNAs. A “change in expression” refers to a difference in the measurement of the biomarkers in a sample and known, controlled measurements of the biomarkers, such as the difference in level of microRNAs expressed of the biomarkers.

The term “HPV” as used herein refers to Human papillomavirus. HPV is a DNA virus from the papillomavirus family that is capable of infecting humans. An oncogenic HPV is an HPV that has a relatively high propensity, in comparison with other types of HPV, to induce the development of cancer over time. The oncogenic potential of human papillovaviruses are known to those skilled in the art. See Madkan et al., British Journal of Dermatology, 157(2):228-241 (2007). Examples of HPV subtypes that turn on oncogenic gene expression include subtypes HPV16 and HPV18.

As used herein, the terms “treatment,” “treating,” and the like, refer to obtaining a desired pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs.

As used herein and in the appended claims, the singular forms “a”, “and”, and “the” include plural referents unless the context clearly dictates otherwise. Thus, for example, reference to “a sample” also includes a plurality of such samples and reference to “the microRNA” includes reference to one or more microRNA, and so forth.

The term “marker” as used herein refers to a microRNA used as an indicator of a biological state or condition, the condition being infection with oncogenic HPV. Changes in the level of expression of microRNA (SEQ ID No.'s 1-13) are associated with oncogenic HPV. SEQ ID No:1, SEQ ID No:2, SEQ ID No:3, SEQ ID No:4, SEQ ID No:5, SEQ ID No:6, SEQ ID No:7, SEQ ID No:8, and SEQ ID No:9, SEQ ID No:10, SEQ ID No:11, SEQ ID No12, and SEQ ID No:13. The SEQ IDs of the microRNA markers useful for indicating the presence of oral oncogenic HPV are defined as in Tablet. The microRNA can be detected or measured by an analytic device such as a kit or a conventional laboratory apparatus, which can be either portable or stationary.

TABLE 1
HPV-associated miRNA and probes

| | | | | Probe |
|---|---|---|---|---|
| SEQ | | | | SEQ |
| ID | miR | | Probe | ID |
| NO. | Sequence | Name | Sequence | NO. |
| 1 | ucuuugguuauc | miR-9- | ucauacagcuag | 14 |
| | uagcuguauga | 5p | auaaccaaaga | |
| 2 | caaagugcucau | miR-20b- | cuaccugcacu | 15 |
| | agugcagguag | 5p | augagcauuug | |
| 3 | cauugcacuugu | miR-25- | ucagaccgaga | 16 |
| | cucggucuga | 3p | caagugcaaug | |
| 4 | caaagugcuguu | miR-93- | cuaccugcacga | 17 |
| | cgugcagguag | 5p | acagcacuuug | |
| 5 | uaaagugcugac | miR- | aucugcacug | 18 |
| | agugcagau | 106b-5p | cagcacuuua | |
| 6 | agcuacaucugg | miR- | acccaguagcc | 19 |
| | cuacugggu | 222-3p | agauguagcu | |
| 7 | aaaagcuggguu | miR-320a | ucgcccucuca | 20 |
| | gagagggcga | | acccagcuuuu | |
| 8 | cgcauccccuag | miR-324- | acaccaaugccc | 21 |
| | ggcauuggugu | 5p | uaggggaugcg | |
| 9 | aauugcacggua | miR-363- | uacagauggau | 22 |
| | uccaucugua | 3p | accgugcaauu | |
| 10 | ucguaccgugag | miR-126- | cgcauuauuac | 23 |
| | uaauaaugcg | 3p | ucacgguacga | |
| 11 | ugagaugaagca | miR-143- | gagcuacagug | 24 |
| | cuguagcuc | 3p | cuucaucuca | |
| 12 | ugagaugaagca | miR-145- | gagcuacagug | 25 |
| | cuguagcuc | 3p | cuucaucuca | |
| 13 | acaguagucugc | miR-100a- | uaaccaaugug | 26 |
| | acauugguua | 3p//miR- | cagacuacugu | |
| | | 199b-3p | | |

One aspect of the invention is directed to polynucleotide probes. The term “probe” as used herein refers to a polynucleotide sequence that will hybridize to a complementary target sequence. In one example, the probe hybridizes to a microRNA sequence. The probes provided herein have nucleotide sequences that have 90% sequence identity to polynucleotide sequences that are the complement of a microRNA having altered expression as a result of HPV infection. These probes (exemplified by SEQ ID No.'s 14-26) can detect microRNA markers. Thus, because SEQ ID No.'s 1-13 are markers of oral oncogenic HPV, the corresponding probes (SEQ ID No.'s 14-26) can be used to detect these markers. A method of detecting HPV or oncogenic HPV can use at least one, at least two, at least three, at least four, at least five, at least six, at least seven, at least eight, at least nine, at least ten, at least eleven, at least twelve, or at least thirteen probes selected from the group consisting of SEQ ID No: 14, SEQ ID No:15, SEQ ID No:17, SEQ ID No:18, SEQ ID No:19, SEQ ID No:20, SEQ ID No:21, SEQ ID No:22, SEQ ID No:23, SEQ ID No:24, SEQ ID No:25, and SEQ ID No:26. In a preferred embodiment, at least four probes are selected from the group consisting of SEQ ID No: 14, SEQ ID No:15, SEQ ID No:17, SEQ ID No:18, SEQ ID No:19, SEQ ID No:20, SEQ ID No:21, SEQ ID No:22, SEQ ID No:23, SEQ ID No:24, SEQ ID No:25, and SEQ ID No:26. The probes and their associated SEQ IDs are provided in Tablet.

Another aspect of the invention provides a method for determining if a subject is infected by an oncogenic HPV that includes the steps of obtaining a sample from the subject; determining the level of a microRNA whose expression is altered in response to infection by an oncogenic HPV, comparing the level of the microRNA to a control level, and determining that the subject is infected by an oncogenic HPV if the level of the microRNA is altered relative to that of the control level. To determine if increased or decreased expression of the microRNA has occurred, the level of microRNA is compared to a control level.

The degree of an increase in the expression level of the present microRNA when determined as indicating the presence of oncogenic HPV can be, for example, preferably 50% or more, more preferably 75% or more, still more preferably 100% or more as a percentage relative to a control, and the degree of a decrease in the expression level of the present microRNA when determined as indicating the presence of oncogenic HPV can be, for example, preferably 25% or more, more preferably 50% or more, still more preferably 75% or more as a percentage relative to a control.

A number of the methods described herein include the step of obtaining a biological sample from the subject. A “biological sample,” as used herein, is meant to include any biological sample from a subject that is suitable for analysis for detection of the microRNA whose level varies in response to infection of the subject by oncogenic HPV. Suitable biological samples include but are not limited to bodily fluids such as blood-related samples (e.g., whole blood, serum, plasma, and other blood-derived samples), urine, sputem, cerebral spinal fluid, bronchoalveolar lavage, and the like. Another example of a biological sample is a tissue sample. The probes can be used to detect oncogenic HPV using samples obtained from a variety of tissue sites. In some embodiments samples are obtained from anal, cervical, and penile tissue. The level of microRNA can be assessed either quantitatively or qualitatively, and detection can be determined either in vitro or ex vivo.

A biological sample may be fresh or stored. Biological samples may be or have been stored or banked under suitable tissue storage conditions. The biological sample may be a tissue sample expressly obtained for the assays of this invention or a tissue sample obtained for another purpose which can be subsampled for the assays of this invention. Preferably, biological samples are either chilled or frozen shortly after collection if they are being stored to prevent deterioration of the sample.

In some embodiments, the sample is an oral rinse sample. The oral rinse can be a liquid. The liquid can be, but is not limited to any of the following: mouthwash, saline rinse, liquid rinse and liquid mixture. In one embodiment, at least about 8 to at least about 35 mL of oral rinse is used. In another embodiment, 10 to 30 mL is used. For example, a volume of 10, 15, 20, 25, or 30 mL is used. In another example, a volume of at least about 8, 9, 10, 11, 12, 13, 14, 15, 16, 17, 18, 19, 20, 21, 22, 23, 24, 25, 26, 27, 28, 29, 30, 31, 32, 33, 34, or 35 mL. In a preferred embodiment, volume is 10 to 20 mL oral rinse. In another preferred embodiment, volume is 13 to 18 mL. This preferred embodiment provides an optimal balance between comfort to the subject while allowing maximum access to the cells at the rinsing and gargling step.

To obtain an oral sample, the oral rinse is swished and/or gargled in a subject's mouth for less than one minute (e.g., 10 to 30 seconds) before being expelled. Swishing is the process of holding an oral rinse in the mouth while moving it using the cheeks and tongue, while gargling is the process of washing one's mouth and throat with a liquid kept in motion by exhaling through it. For example, the oral sample can be swished and/or gargled 10, 15, 20, 25, or 30 seconds. In another example, the oral sample can be swished and/or gargled 8, 9, 10, 11, 12, 13, 14, 15, 16, 17, 18, 19, 20, 21, 22, 23, 24, 25, 26, 27, 28, 29, 30, 31, 32, 33, 34, or 35 seconds. In a preferred embodiment, the oral sample is swished and/or gargled is 23 to 33 seconds. This preferred embodiment provides the advantage of optimal time of swishing and gargling in the mouth to obtain the maximum number of cells for detection analysis. In an alternate embodiment, the sample could be taken as a cervical sample. In an additional alternate embodiment, the sample could be taken as an anal sample.

A person skilled in the art will appreciate that a number of methods can be used to detect or quantify the level of microRNA biomarkers within a sample, including microarrays, PCR (including quantitative RT-PCR), nuclease protection assays, in situ hybridization, and microfluidics devices. In one embodiment, the assay used is PCR, as described in Example 3 which uses quantitative RT-PCR.

The microarray method is not particularly limited provided that it can measure the level of the microRNA whose expression changes in response to infection by oncogenic HPV; examples thereof can include a method which involves labeling the RNA extracted from a tissue with a label (preferably a fluorescent label), contacting the RNA with a microarray to which a probe consisting of a polynucleotide (preferably DNA) consisting of a nucleic acid sequence complementary to the microRNA to be identified or a part thereof is fixed for hybridization, washing the microarray, and measuring the expression level of the remaining microRNAs on the microarray.

The type of the nucleotide of the nucleic acid sequence is not particularly limited provided that it can specifically hybridize to the microRNA of the present invention. The length of the part of the polynucleotide is not particularly limited provided that it specifically hybridizes to the predetermined microRNA according to the present invention; however, it is preferably 10 to 100 mers, more preferably 10 to 40 mers in view of securing the stability of hybridization. The polynucleotide or a part thereof can be obtained by chemical synthesis or the like using a method well known in the art.

The array to which the polynucleotide or a part thereof is fixed is not particularly limited; however, preferred examples thereof can include a glass substrate and a silicon substrate, and the glass substrate can be preferably exemplified. A method for fixing the polynucleotide or a part thereof to the array is not particularly limited; a well-known method may be used.

The quantitative PCR method is not particularly limited provided that it is a method using a primer set capable of amplifying the sequence of the microRNA and can measure the expression level of the present microRNA; conventional quantitative PCR methods such as an agarose electrophoresis method, an SYBR green method, and a fluorescent probe method may be used. However, the fluorescent probe method is most preferable in terms of the accuracy and reliability of quantitative determination.

The primer set for the quantitative PCR method means a combination of primers (polynucleotides) capable of amplifying the sequence of the microRNA. The primers are not particularly limited provided that they can amplify the sequence of the microRNA; examples thereof can include a primer set consisting of a primer consisting of the sequence of a 5′ portion of the sequence of a microRNA of the present invention (forward primer) and a primer consisting of a sequence complementary to the sequence of a 3′ portion of the microRNA (reverse primer). Here, the 5′ means 5′ to the sequence corresponding to the reverse primer when both primers were positionally compared in the sequence of a mature microRNA; the 3′ means 3′ to the sequence corresponding to the forward primer when both primers were positionally compared in the sequence of a microRNA.

Preferred examples of the 5′ sequence of a microRNA can include a sequence 5′ to the central nucleic acid of the microRNA sequence; preferred examples of the 3′ sequence of the microRNA can include a sequence 3′ to the central nucleic acid of the microRNA sequence. The length of each primer is not particularly limited provided that it enables the amplification of the microRNA; however, each primer is preferably a 7-to-10-mer polynucleotide. The type of the nucleotide of a polynucleotide as the primer is preferably DNA because of its high stability.

In some embodiments, a fluorescent probe is used. The fluorescent probe is not particularly limited provided that it comprises a polynucleotide consisting of a nucleic acid sequence complementary to the sequence of the present microRNA or a part thereof; preferred examples thereof can include a fluorescent probe capable of being used for the TaqMan™ probe method or the cycling probe method; the fluorescent probe capable of being used for the TaqMan™ probe method can be particularly preferably exemplified. Examples of the fluorescent probe capable of being used for the TaqMan™ probe method or the cycling probe method can include a fluorescent probe in which a fluorochrome is labeled 5′ thereof and a quencher is labeled on 3′ thereof. The fluorochrome, quencher, donor dye, acceptor dye used or the like used with a fluorescent probe are commercially available.

In some embodiments, the level of microRNA can be determined using a microfluidic chip. Use of a microfluidic chip includes the steps of making an assay mixture containing at least one microRNA; providing a microchamber electrochemical cell comprising a substrate defining a pair of opposing microchannels; at least one ion exchanging nanomembrane coupled between the opposing microchannels such that the microchannels are connected to each other only through the nanomembrane; wherein said at least one polynucleotide is selected from the group consisting of SEQ ID No:14, SEQ ID No:15, SEQ ID No:16, SEQ ID No:17, SEQ ID No:18, SEQ ID No:19, SEQ ID No:20, SEQ ID No:21, and SEQ ID No:22; a device for measuring the electrical current of potential across the nanomembrane; flowing the assay mixture through the opposing microchannels; and detecting a change in the measure electrical current of potential across the nanomembrane to qualify the presence of the microRNA.

Conventional techniques of molecular biology, microbiology and recombinant DNA techniques, are within the skill of the art. Such techniques are explained fully in the literature. See, e.g., Sambrook, Fritsch & Maniatis, 1989, Molecular Cloning: A Laboratory Manual, Second Edition; Oligonucleotide Synthesis (M. J. Gait, ed., 1984); Nucleic Acid Hybridization (B. D. Harnes & S. J. Higgins, eds., 1984); A Practical Guide to Molecular Cloning (B. Perbal, 1984); and a series, Methods in Enzymology (Academic Press, Inc.); Short Protocols In Molecular Biology, (Ausubel et al., ed., 1995).

A person skilled in the art will appreciate that a number of detection agents can be used to determine the expression of the biomarkers. For example, to detect microRNA biomarkers, probes, primers, complementary polynucleotide sequences or polynucleotide sequences that hybridize to the microRNA products can be used. In some embodiments, reverse complementary poylynucleotides serve as probes for microRNA markers. In alternate embodiments, a complementary polynucleotide sequence that hybridizes to the target polynucleotide sequence can be used to detect expression of the microRNA markers.

Another aspect of the invention provides a method of treating oncogenic HPV infection in a subject in need thereof that includes the steps of obtaining a biological sample from the subject, determining if microRNA associated with the presence of oncogenic HPV in the biological sample shows a change in expression relative to controls, and providing treatment of oncogenic HPV infection for subjects identified as exhibiting a change in expression levels of the microRNA. Methods of treating infection by oncogenic HPV include treatment with antiviral agents, or if precancerous growth is present, treating the precancerous cells with cryotherapy, conization, or Loop Electrosurgical Excision Procedure (LEEP).

### Kits

The present disclosure also provides kits for detecting oncogenic human papillomavirus in a subject. The kits include one or more primers and/or probes capable of hybridizing with microRNA associated with oncogenic HPV, and a package for holding the primers or probes. A kit generally includes a package with one or more containers holding the reagents, as one or more separate compositions or, optionally, as an admixture where the compatibility of the reagents will allow. The kits may further include enzymes (e.g., polymerases), buffers, labeling agents, nucleotides (dNTPs), controls, and any other materials necessary for carrying out the detection of oncogenic HPV. Kits can also include a tool for obtaining a sample from a subject, such as a suitably sized vessel for providing and receiving an oral sample.

In an exemplary embodiment, detection kits comprising polynucleotides attached or immobilized to a solid support. In another embodiment, detection kits are based on a hybridization assay. In a further embodiment, detection kits are based on a reverse hybridization assay.

The kit for the oncogenic HPV may further comprise any element, such as reagents used for a microarray method, including, for example, a reagent used for RNA-labeling reaction, a reagent used for hybridization, a reagent used for washing, and a reagent used for extracting RNA from a tissue in addition to the above-described microarray. The microarray method can be specifically exemplified by a method which involves measuring the expression level of a microRNA on DNA Microarray Scanner (from Agilent Technologies) using Agilent Human miRNA V2 (from Agilent Technologies) according to the method described in Agilent Technologies' miRNA Microarray Protocol Version 1.5. The microarray to which the probe consisting of the polynucleotide or a part thereof is fixed can be prepared, for example, by synthesizing a polynucleotide based on the sequence information of the present microRNA to be measured and fixing it to a commercially available array.

In some embodiments, a kit comprises one or more pairs of primers (a “forward primer” and a “reverse primer”) for amplification of a cDNA reverse transcribed from a target RNA for carrying out PCR or RT-PCR. Accordingly, in some embodiments, a first primer comprises a region of at least 4, at least 5, at least 6, at least 7, at least 8, at least 9, or at least 10 contiguous nucleotides having a sequence that is identical to the sequence of a region of at least 4, at least 5, at least 6, at least 7, at least 8, at least 9, or at least 10 contiguous nucleotides at the 5′-end of a target RNA. Furthermore, in some embodiments, a second primer comprises a region of at least 4, at least 5, at least 6, at least 7, at least 8, at least 9, or at least 10 contiguous nucleotides having a sequence that is complementary to the sequence of a region of at least 4, at least 5, at least 6, at least 7, at least 8, at least 9, or at least 10 contiguous nucleotides at the 3′-end of a target RNA. In some embodiments, the kit comprises at least a first set of primers for amplification of a cDNA that is reverse transcribed from a target RNA capable of specifically hybridizing to a nucleic acid comprising a sequence identically present in one of SEQ ID NOs: 1 to 13 and/or a cDNA that is reverse transcribed from a target RNA.

In some embodiments, the kit comprises at least two, at least four, at least 10, or at least 13 sets of primers, each of which is for amplification of a cDNA that is reverse transcribed from a different target RNA capable of specifically hybridizing to a sequence selected from SEQ ID NOs: 1 to 13 and/or a cDNA that is reverse transcribed from a target In some embodiments, the kit comprises at least one set of primers that is capable of amplifying more than one cDNA reverse transcribed from a target RNA in a sample.

In some embodiments, probes and/or primers for use in the compositions described herein comprise deoxyribonucleotides. In some embodiments, probes and/or primers for use in the compositions described herein comprise deoxyribonucleotides and one or more nucleotide analogs, such as LNA analogs or other duplex-stabilizing nucleotide analogs described above. In some embodiments, probes and/or primers for use in the compositions described herein comprise all nucleotide analogs. In some embodiments, the probes and/or primers comprise one or more duplex-stabilizing nucleotide analogs, such as LNA analogs, in the region of complementarity.

In some embodiments, the kits for use in RT-PCR methods described herein further comprise reagents for use in the reverse transcription and amplification reactions. In some embodiments, the kits comprise enzymes such as reverse transcriptase, and a heat stable DNA polymerase, such as Taq polymerase. In some embodiments, the kits further comprise deoxyribonucleotide triphosphates (dNTP) for use in reverse transcription and amplification. In further embodiments, the kits comprise buffers optimized for specific hybridization of the probes and primers.

The kit can also include instructions for using the kit to carry out a method of detecting oncogenic HPV in a subject. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

Examples have been included to more clearly describe a particular embodiment of the invention and its associated cost and operational advantages. However, there are a wide variety of other embodiments within the scope of the present invention, which should not be limited to the particular examples provided herein.

### EXAMPLES

### Example 1

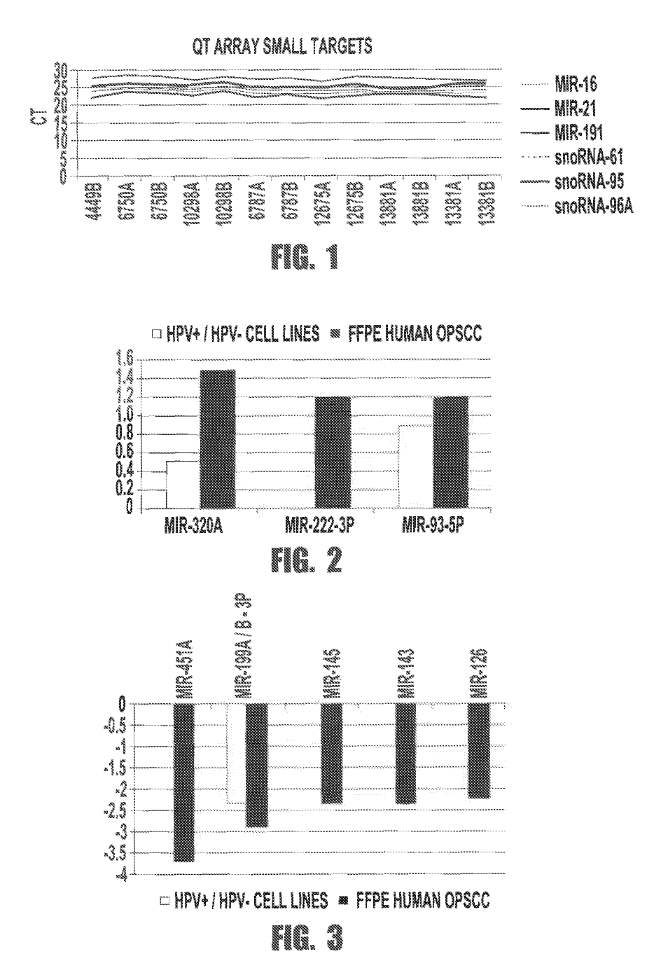

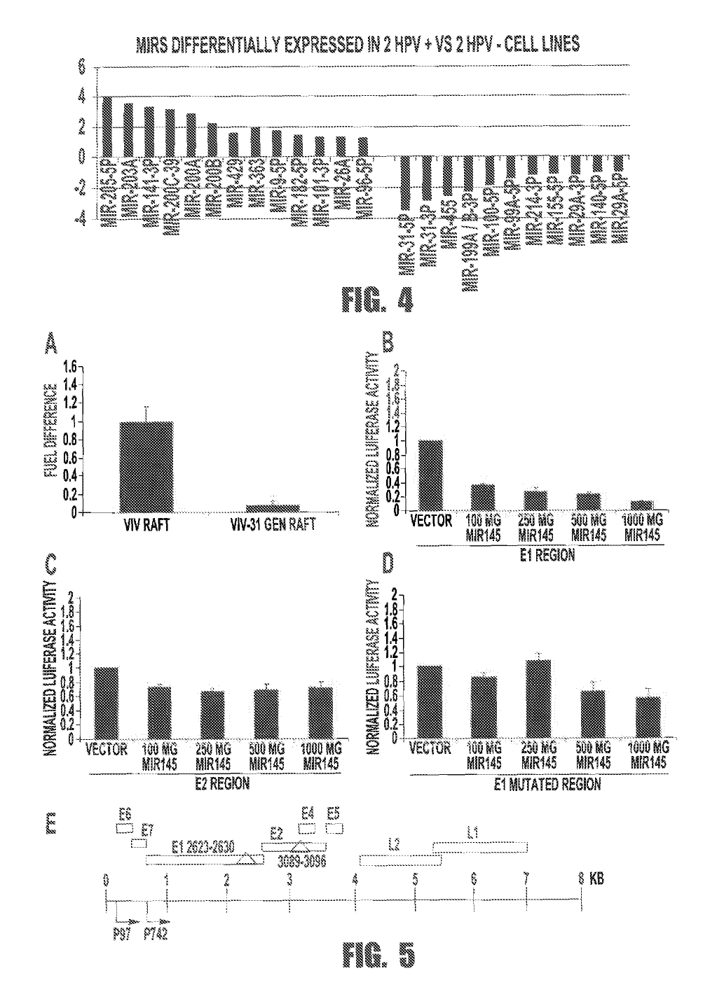

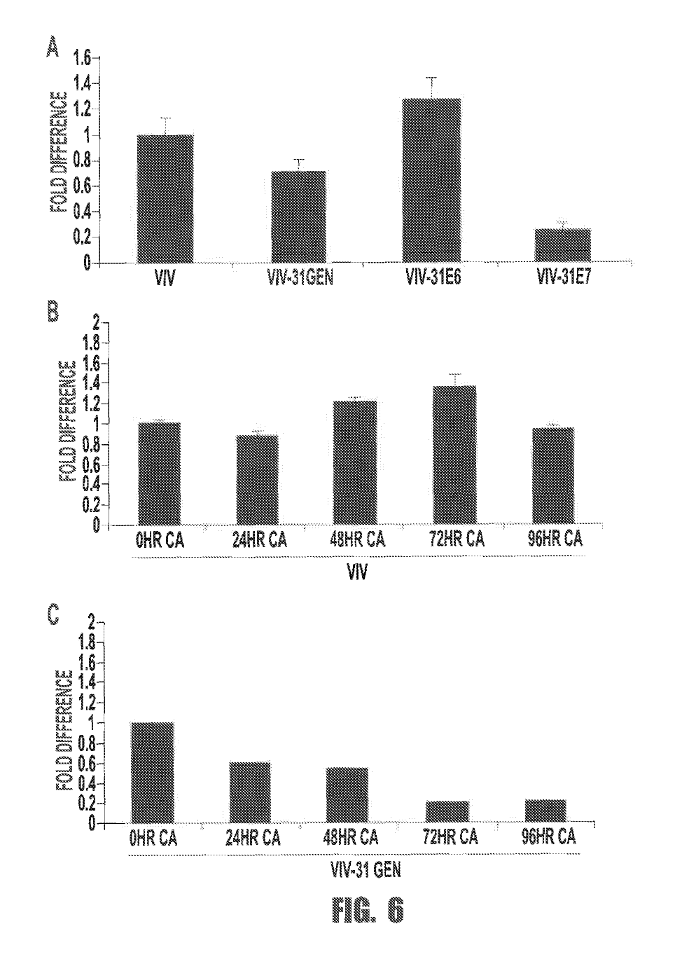

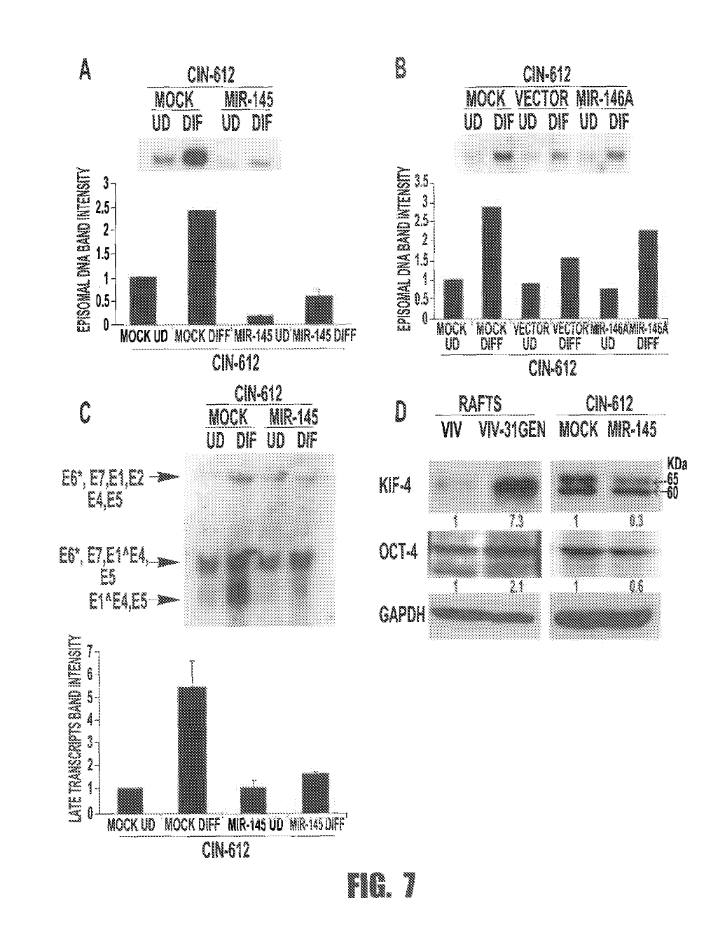

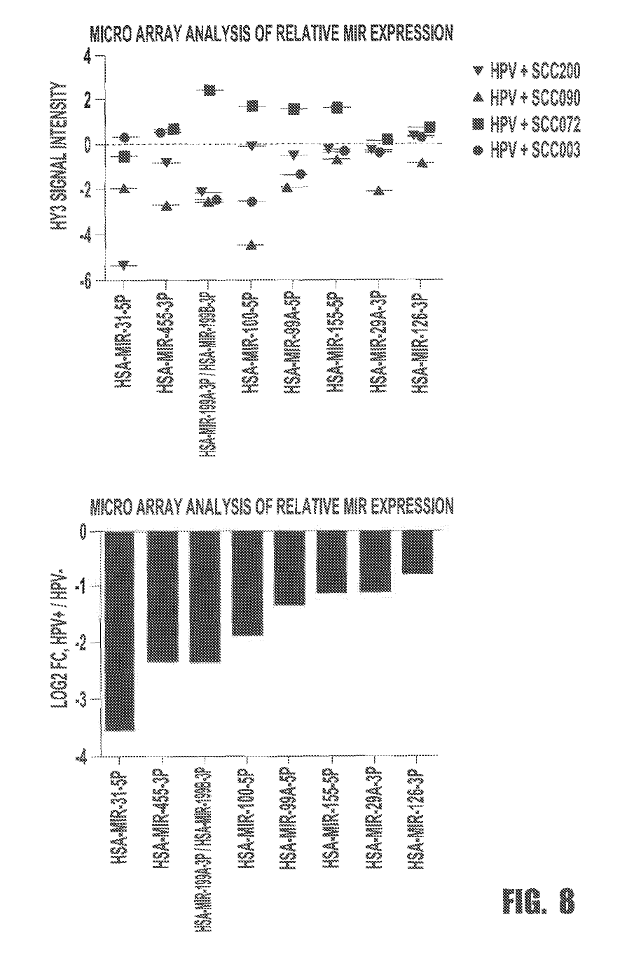

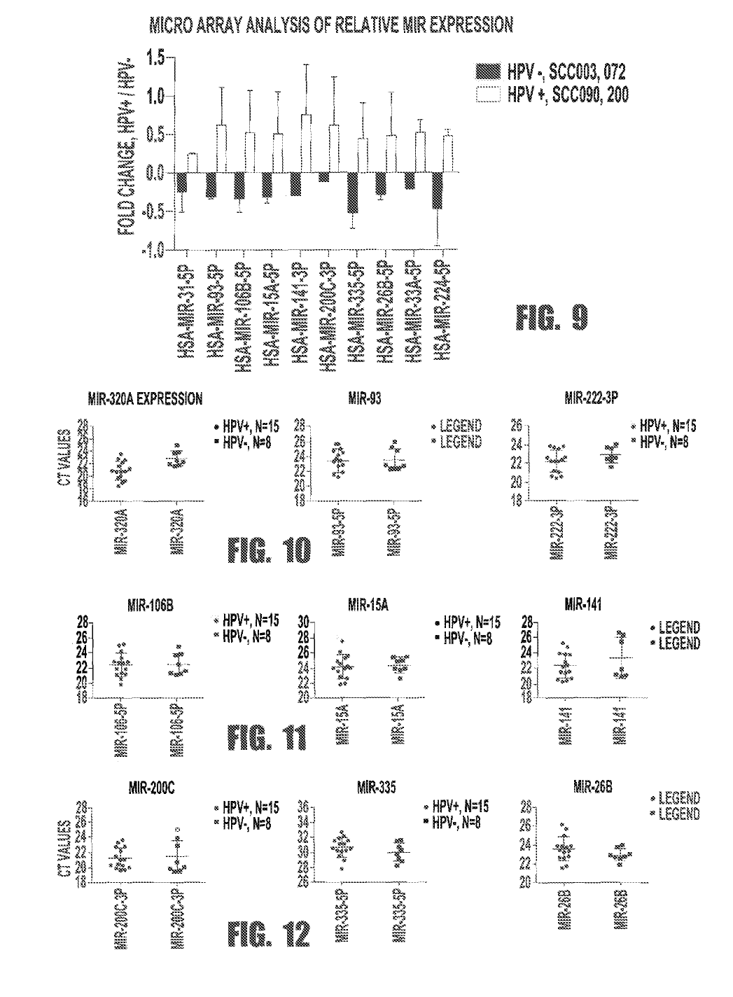

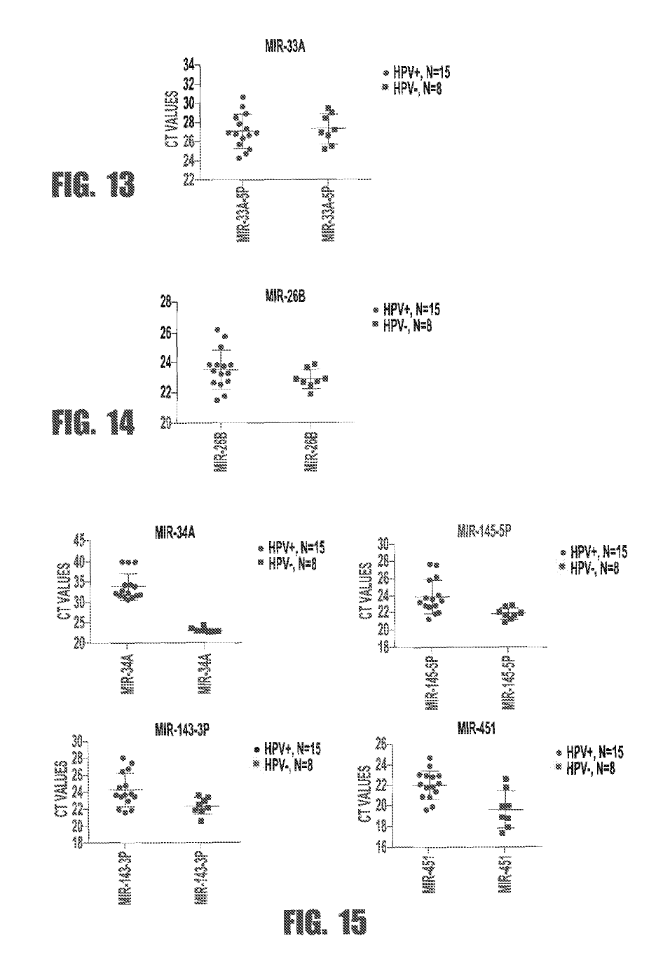

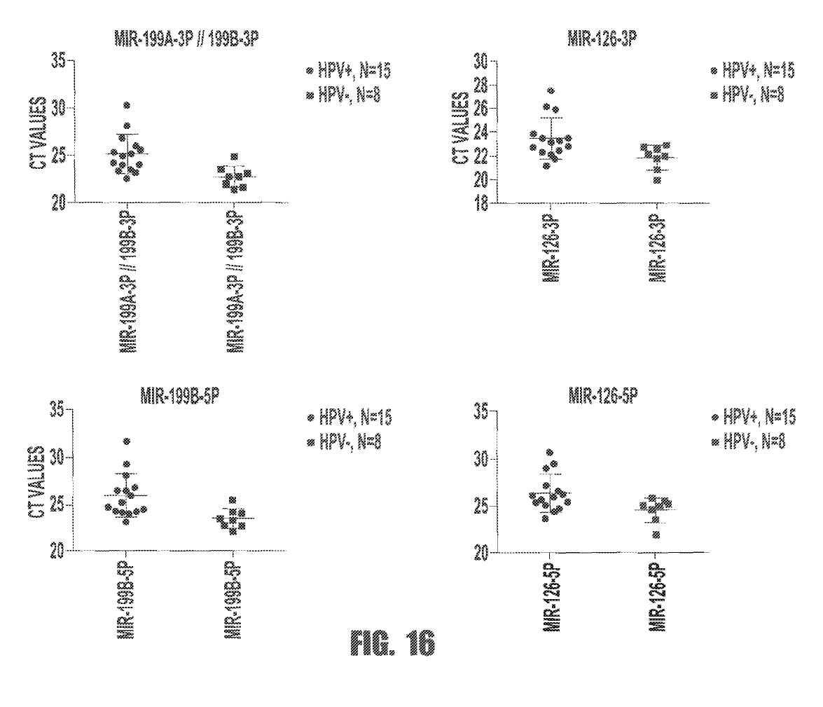

In operation, in an exemplary embodiment an in vivo orthotropic model of aggressive, poorly differentiated tongue squamous cell carcinoma was established and used to perform miRNA array profiling to identify microRNAs important in disease progression. These results identified 29 differentially expressed miRNAs (18 increased, 11 decreased), two of which were validated by qPCR: miR-146a (6-fold down-regulation) and miR-452 (7-fold upregulation). Expression of miR-146a was low in 3 distinct aggressive oropharyngeal squamous cell carcinoma (OSCC) cell lines relative to normal oral epithelium. Because these universally expressed non-coding RNA are stable across samples, it is concluded that miRNA is relatively stable and protected in paraffin, as shown in FIG. 1. Therefore, one can detect biologically relevant differences in miRNA between samples. This was validated by fluorescent in situ hybridization analysis of formalin-fixed paraffin-embedded (FFPE) human tissues showing loss of miR-146a. Restored expression of miR-146a using lentiviral vectors that do not result in supra-physiological expression levels and have shown that restoration of miR-146a results in an 8-fold decrease in invasion in vitro and reduced tumor growth in vivo. Results of up-regulated are shown in Table 2. Results of down-regulated are shown in Table 3. The results are graphically represented in FIGS. 2 and 3.

TABLE 2

| “UP” | logFC | FC | P-value |
|---|---|---|---|
| miR-320a | 1.50 | 2.83 | 0.002 |
| miR-222-3p | 1.20 | 2.31 | 0.004 |
| miR-93-5p | 1.24 | 2.36 | 0.005 |

With regard to miR-320a, the microarray data from 6 OPSCC cell lines supports upregulation in HPV+ cell lines compared to HPV−. A proposed role for miR-320a may be in affecting B-catenin, and may act in a redundant manner with the miR-200 cluster (significantly up regulated in HPV+ cell lines). With regard to miR-222, this miR is a family member with miR-221, may regulate radiosensitivity, and cell growth and invasion, and includes p27kip1 as a validated target. MiR-93-5p was upregulated in 2 cervical cancer studies, and is a member of the miR-106b cluster, which likely has considerable biological redundancy with the miR-17-92 cluster.

TABLE 3

| “DOWN” | logFC | FC | P-value |
|---|---|---|---|
| miR-451a | −3.7 | 0.07 | 0.003 |
| miR-199a-3p// | −2.86 | 0.13 | 0.005 |
| 199b-3p | | | |
| miR-199b-5p | −2.85 | 0.13 | 0.008 |
| miR-145-5p | −2.96 | 0.20 | 0.005 |
| miR-143-3p | −2.27 | 0.20 | 0.002 |
| miR-126-5p | −2.14 | 0.22 | 0.005 |
| miR-126-3p | −1.93 | 0.26 | 0.006 |

With regard to the downregulated miR, miR-451 is significantly upregulated in the saliva of patients with esophageal SCC1. The role of miR-199a is unclear, but it is most likely involved in modulating metastatic genes, and was shown in one study to be downregulated in both cervical and HNSCC2. miR-143/145 was down-regulated in three cervical cancer studies and at least one HNSCC study, and has been shown to be a tumor suppressor (and downregulated) in esophageal SCC3. miR-126 likely modulates Pi3K/Akt/mTOR, and was down-regulated in 3 studies in cervical cancer

In an exemplary embodiment, a cohort of samples were collected, using the following protocol. A new pair of nitrile or latex gloves was used for each sample collection, Scope® mouthwash, Saline solution used as an alternative to Scope for participants with oral ulcers or those unable to tolerate mouthwash, and a 2 ounce sterile medicine cup for dispensing mouthwash. Also needed was a 5 ounce sterile specimen container to spit the mouthwash sample into and store the sample. A bar-code label was applied to each specimen container with a de-identified number code to catalog the sample while maintaining confidentiality. Finally, a consent form, a cooler with ice for sample storage, and a timer or stopwatch were used. Participants aged eighteen and older were eligible for the oral rinse component. There were no additional exclusion criteria for this exam. Gloves were put on and mouthwash was poured into a medicine cup, making sure not to touch the rim of the cup. The medicine cup with the mouthwash was handed to the participant. When the participant was ready, she/he was instructed to put mouthwash in mouth and swish for 15 seconds. After 15 seconds, the participant was instructed to begin gargling for 15 seconds. When time was up, the specimen container was opened and handed to the participant. The top was held lid-down to avoid contamination. The participant spit the mouthwash into the specimen container. The specimen container was taken from the participant without touching the rim. The specimen container was sealed properly to avoid leakage. The bar-code label was placed on the specimen container. In a preferred embodiment, the subject has consumed any food or beverage in the hour before the sample was collected.

The fluid sample was transferred into a 50 mL conical tube with corresponding barcode number. Using automatic auto cell counter, background count and cell number count was obtained from each sample. 0.5 mL of the oral rinse sample was mixed into 4.5 mL buffer solution. The tubes were centrifuged at 40,000 rpm for five minutes. The appropriate balance from the centrifuge was verified. The supernatant fluid was removed and aliquoted into nine barcoded cryo vials. Each barcode was scanned into database, matching with corresponding sample barcode number. Cryo vials were then stored in liquid nitrogen freezer at −81 degrees. The cell plug was washed with 20 mL of PBS three times. The cells were centrifuged at 1200 rpm for 2 minutes. The liquid was then discarded and cells were re-suspended to reach approximately 10,000 cells per mL. The cytospin centrifuge was set up with Cytopro chamber cups and white absorption pad. Six slides were labeled with barcode numbers and slide a, b, c as needed. Each sample has six slides for cytospin. 0.3 mL of sample was placed into small well of cytopro chamber. The centrifuge was run at 1000 rpm for 5 minutes. At least one aliquot of the cell pellet from each sample was frozen in small conical tubes and placed in the liquid nitrogen freezer at −81° Celsius for later analysis. Slides were removed and discarded cytopro chambers and absorption pads were discarded, also. Slides were fixed in 4% PFA (stock was 16%/10 mL, mixed with 30 mL PBS) for 10 to 20 minutes. Slides were washed with PBS for 2 minutes. Once they were dried, slides were stored in slide box at −81° Celsius freezer in Tissue Core.

### Example 2

In an exemplary embodiment, validation of microRNAs occurs in a three-fold process. First, PCR is conducted on HPV+ compared to HPV− cases with 24 patients. Next, the model “miR expressions=HPV+HPV (smokers)+age” was used to determine the likelihood of miR expression based on the three factors of HPV, smoking, and age. Third, a bioinformatics analysis was performed using publically available databases, such as The Cancer Genome Atlas (TCGA), for example. To test which miRNAs were differentially expressed based on HPV status (for the FFPE PCR profiling), a linear modeling framework designed for high-throughput biological experiments was used. Specifically, using the R/Bionconductor limma package, moderated t-statistics were applied to each microRNA using an Empirical Bayes approach, in which the standard errors are shrunk towards a common value (Smyth G K 2005 & Smyth, G K 2004). The comparison of interest was HPV+vs. HPV−, expressed in terms of fold change (HPV+/HPV−). The comparison was made by adjusting for smoking, smoking and HPV interaction, and age to account for potential confounding effects and expressed as the equation: miRexp=smoking+HPVstatus+smoking*HPVstatus (interaction)+age. One such cohort is shown in Tables 4a and 4b.

TABLE 4a

| | | | | | | Family | Primary or |
|---|---|---|---|---|---|---|---|
| Cell line ID | Age | Gender | Ethnicity | Smoking | Alcohol | Hx | Recurrence |
| SCC003 | 65 | F | Caucasian | Y | Y | N | NP |
| SCC072 | 61 | F | Caucasian | Y | Y | Y | NP |
| SCC090 | 46 | M | Caucasian | Y | Y | Y | R |
| SCC200 | 74 | M | Caucasian | Y | Y | N | NP |

TABLE 4b

| | | | Path | 11q13 | TP53 | |
|---|---|---|---|---|---|---|
| Cell line ID | Site | Grade | Stage | amp. | codon | HPV+ |
| SCC003 | TONS | 1 | T1NO | Y | WT | — |
| SCC072 | TONS | 2 | T2N2B | Y | Mut | — |
| | | | | | (179) | |
| SCC090 | BOT | 3 | T2NO | N | WT | + |
| SCC200 | TONS | 1 | T2N2B | N | WT | + |

### Example 3

In an exemplary embodiment, quantitative reverse transcriptase polymerase chain reaction (qRT-PCR) is used as a detection technique. The objective was to validate the loss or gain of the most differentially expressed microRNAs utilizing (i) in vitro cell line systems designed to recapitulate salient phenotypic features of OPSCC and (ii) an expanded human tissue cohort. In an alternate embodiment, an expanded human saliva sample cohort could be used. Total RNA was extracted from various cell lines using the miRCURY™ RNA Isolation Kit—Cell and Plant (Product number 300110, Exiqon A/S, Denmark). All cells were taken at 50% confluence on 10 cm plates as recommended by the manufacturer.

RNA from 10 cell lines (SCC200, 090, 036, 152, HTE clone 21505, SCC003, 072, 089, 103, and HTE clone D) in technical replicates of three, was extracted utilizing miRCURY RNA Isolation kit (Exiqon) according to the manufacturer's instructions for a total of 30 RNA samples. This kit utilized spin column chromatography using a resin to separate total RNA, including mRNA, rRNA, and other small RNAs, from other cell components without the use of phenol, trizol, or chloroform. Each of the 30 samples was polyadenylated and reverse transcribed into complementary DNA in a single reaction with the Universal cDNA Synthesis kit II (Exiqon). A synthetic RNA spike-in, UniSp6, was added to each sample as a means to monitor RT efficiency and reproducibility in the final qPCR experiments.

Pick-&-Mix microRNA PCR Panel (Exiqon, product #203801 and 203802) consist of 96-well PCR plates containing custom selections of dried down microRNA LNA™ PCR primer sets for one 10 μL real-time PCR reaction per well, ready-to-use. The LNA™ primer sets are designed for optimal performance with the Universal cDNA Synthesis Kit II and the ExiLENT SYBR® Green master mix kit. Primer sets for 18 microRNAs of interest were selected based on microarray data and the FFPE PCR profile. Also included, were 4 candidate reference genes and 2 positive control primer sets, for a total of 8 plates. Each of the 8 plates thus contained wells to assay 24 PCR reactions for 4 samples. The 30 samples were divided into groups of 4 different biological replicates such that cDNA from a single cell line was dispersed between 3 plates. Two negative control samples were included on plate 8, one no template control (NTC) and one sample that was run without reverse transcription (No Enzyme control).

Prior to dispensing samples across the plates, cDNA from the RT reactions were diluted 100× with nuclease free water. cDNA and 2×PCR master mix (Exiqon) were combined 1:1 and 10 mcls added to each well, corresponding to 0.05 ng total RNA per PCR reaction. The plate was then sealed as recommended by the PCR instrument manufacturer and spun in a plate centrifuge for 1 minute.

Real-time PCR Amplification and melting curve analysis was performed on an ABI Step One Plus in a standard (2 hr) run according to the following cycles: (1) polymerase activation/denaturation at 95° C. for 10 minutes, (2) amplification for 40 cycles at 95° C. for 10 seconds followed by 60° C. for 1 minute at a ramp-rate of 1.6° C./s, and (3) melting curve analysis as specified by the StepOne Plus system. The ABI system was set to manual baseline and threshold as opposed to auto Ct settings as per the recommendation of Exiqon. Raw Ct values, amplification, plate setup, melt region temperature, melt region normalized, and melt region derivative data were exported as .txt and .xls files for data analysis.

The results showed an upregulation of SEQ IDs 1-9, and a down-regulation of SQ IDs 10-13 in samples of subjects with oncogenic HPV. FIG. 5 demonstrates the results of miR-145. The miR-145 sequence in E1 showed a significant reduction (80%) in luciferase activity with increasing levels of miR-145 (B), whereas the E2 region showed a slight reduction in luciferase activity (C). The data are from three independent experiments, and standard errors are shown.

The results of the HPV+/HPV− comparison were first sorted according to adjusted p-value, which is the Benjamini-Hochberg false discovery rate (fdr) adjustment that accounts for the large number of tests performed, as shown in Table 5. For this, an fdr=0.2 was chosen since validation studies were planned, although 0.3 or 0.4 would have been considered acceptable given the planned validation efforts. Moderated T tests were performed to produce p-values for each microRNA comparing HPV+ to HPV−.

TABLE 5

| | | | Adj. P. Val | |
|---|---|---|---|---|
| ID | logFC | P. Value | (aka FDR) | PC_positive |
| miR-320a | 1.501044432 | 0.002024594 | 0.20380063 | 2.830475493 |
| miR-143-3p | −2.277057625 | 0.002502535 | 0.20380063 | 0.206318111 |
| miR-451a | −3.705247757 | 0.003195508 | 0.20380063 | 0.076667144 |
| miR-222-3p | 1.205682839 | 0.004577297 | 0.20380063 | 2.306464103 |
| miR-93-5p | 1.237836139 | 0.005405395 | 0.20380063 | 2.358445298 |
| miR-199a-3p// | −2.866705587 | 0.005316526 | 0.20380063 | 0.137099424 |
| miR-199b-3p | | | | |
| miR-126-5p | −2.142609506 | 0.005480287 | 0.20380063 | 0.226469786 |
| miR-145-5p | −2.296799107 | 0.005907265 | 0.20380063 | 0.203514133 |
| miR-126-3p | −1.939807196 | 0.006935735 | 0.212695871 | 0.260651272 |
| miR-199b-5p | −2.850689997 | 0.008802817 | 0.242957761 | 0.138629866 |

### Example 4

In an exemplary embodiment, a microfluidics device is used as a detection technique. 15 mL of a saliva sample, collected from a human subject, was run through a filter (from Milipore, Code VVLP, size 100 nm), separating all cells. The filtered cells were then collected. 50 μL of lysis buffer, (BP-200 from Boston Bioproducts) was added to the filtered cells, and the cells are lysed. Then, 50 μL of glycerol (from SigmaAldrich) was added to the solution. Following the addition of glycerol, 100 μL of this lysate was pipetted onto the microfluidics chip. All electrodes and fluidic connections were placed at their positions, and reservoirs on the chip were filled with buffer. An electrochemical baseline of the sensor is measured. Next, an extraction of the target microRNAs from the lysate took place by applying an electrical field. The extracted target microRNAs were concentrated at the sensor by applying convective flow and an electrical field on the system. The target molecules hybridized on the short RNA probes on the sensor. All non-target molecules were washed off the sensor. An electrochemical measurement of the sense was conducted to determine the change compared to the baseline. The size of the change indicated the concentration of target molecules in the sample.

The results showed a change in the output electrochemical curve, shifting the baseline curve to the right, indicating the presence of the target microRNAs.

### Example 5

In an exemplary embodiment, in situ hybridization (ISH) is used as a detection technique. For the tissue-based in situ hybridization studies, the aim was to assess the expression of microRNAs that were statistically significant between HPV+vs HPV− oropharyngeal squamous cell carcinoma (OSCC) and expressed at relatively high levels according to miRNAseq data utilizing (i) in vitro cell line systems designed to recapitulate salient phenotypic features of OPSCC and (ii) an expanded human tissue cohort. In an alternate embodiment, an expanded human saliva sample cohort could be used.

Head and neck squamous cell carcinoma (HNSCC) tissue microarrays (TMA) were provided by Dr Jim Lewis (Department of Pathology & Immunology, Washington University School of Medicine, St Louis, Mo.). The TMA included 357 cases of HNSCC with two tumor tissue cores per case, thus compromised 17 formalin-fixed paraffin embedded tissue blocks (FFPE). Each block was sectioned onto four separate slides (4 μm thick), for a total of 68 slides and sent to The Center for RNA interference and Non-Coding RNAs at MD Anderson Cancer Center (MDACC, Houston, Tex.) for ISH hybridization and staining. The probes for in situ hybridization (ISH) were ordered from Exiqon (Denmark) and shipped directly to MDACC. The probes were selected based on the results of FFPE, TCGA, and cell line profiling based on their high relative expression levels and statistically significant differences between HPV+ and HPV− tumor tissues. Double digoxigenin (DIG) labeled (5′ and 3′) miRCURY LNA™ probes are optimized for detection of microRNAs in FFPE tissue sections. For visualization, the digoxigenins are detected with a polyclonal anti-DIG antibody and alkaline phosphatase-conjugated secondary antibody using 5-bromo-4-chloro-3-indolyl-phosphate/nitro blue tetrazolium (BCIP/NBT).

In these experiments, the full-length mature microRNA sequences were used for three specific locked nucleic acid (LNA) probes:

SEQ ID No.: 27
TCATACAGCTAGATAACCAAAGA;,
SEQ ID No.: 28
ATCTGCACTGTCAGCACTTTA;,
SEQ ID No.: 29
GAACAGGTAGTCTGAACACTGGG,

Optimization of binding conditions was performed by MDACC, but briefly the tissue is first digested briefly with Protease K. Next, the probes are hybridized to their targets. Followed by AP-conjugated antibodies and BCIP/NBT development to produce purple signal. All tumor tissues stained for the three microRNAs were counterstained with nuclear fast red. One entire TMA was stained with LNA U6 snRNA probes as positive controls without counterstain. Each TMA contained normal reference tissues that served as negative controls (liver, thyroid, and small bowel).

The stained slides were shipped, read, and scored in a systematic fashion according to the following 3 parameters: (1) proportion of tumors cells with identifiable ISH signal (0<10%, 1=10-25%, 2=25-50%, 3=50-75%, 4=>75%); (2) strength of ISH signal (weak, weak/moderate, moderate, strong); (3) staining pattern (punctate, diffuse, or punctate+diffuse). All images were assessed in a blinded fashion as to HPV status, H&E morphology, and all other clinicopathological parameters.

The staining results were tabulated on a .csv file and then compared to prior data concerning HPV status. The subsequent analysis was performed. Logistic regression analysis was performed separately for each binary outcome (HPV status measured by p16 and ISH) with the following for predictors: staining proportion, strength of ISH signal, and staining pattern. Model selection criteria were used to guide the selection of the final variables included in the model. Pseudo-R² and internal cross validation were reported to indicate the performance of the model.

Results showed hybridization of the probes to microRNA in the samples of subjects with oncogenic HPV. Those samples not infected with oncogenic HPV did not hybridize to the probes.

### Example 6

### Identification of Human Papillomavirus-Associated Oncogenic microRNA Panel in Human Oropharyngeal Squamous Cell Carcinoma Validated by Bioinformatics Analysis of the Cancer Genome Atlas

Expression profiling of cancers and functional studies performed in cancer cell lines and murine models have revealed provocative patterns implicating miRNA-mRNA dysregulation as important in tumor development and progression. Somewhat independent of miRNA biology, miRNA profiles offer diagnostic and prognostic utility in cancer and other diseases that may guide treatment. Schetter et al., JAMA., 299(4): 425-36 (2008); Schultz et al., JAMA., 311(4):392-404 (2014). To this end, the objective of this study was to profile HPV+ versus HPVOPSCC to provide a more detailed understanding of pathological molecular events and to identify biomarkers that may have applicability for early diagnosis, improved staging, and prognostic stratification. We identify an “oncogenic miRNA panel” that represents the host response to an oncogenic HPV infection and validate this panel in additional clinical cohorts using publicly available sequencing and clinical data from The Cancer Genome Atlas (TCGA) and miRNA-in situ hybridization (miRNA-ISH) analysis of arrayed human OPSCC tissues. This molecular signature may have utility to differentiate oropharyngeal tumors with different prognoses and thus distinct management strategies and facilitate mechanistic elucidation of molecular factors that contribute to OPSCC development, progression and response to therapy.

Materials and Methods

Patient Samples for miRNA Profiling. Tissues for the initial study cohort were obtained from University of Missouri surgical pathology archives (2006-2011) with Institutional Review Board approval and represent histologically confirmed tonsillar or base of tongue squamous cell carcinoma (OPSCC). Tissue for study was identified by staining for p16 according to the manufacturer's instructions (CINtec Histology Kit, MTM laboratories; E6H4 clone; Ventana Medical Systems, Roche) and evaluated using a binary rating system, with positive representing extensive (>50%) tumor-cell specific cytoplasmic and nuclear staining. Negative staining represented sparse or absent tumor-specific staining. ‘Focal staining patterns’, in the presence of mostly negative staining was interpreted as negative. All cases included for laser capture microdissection represented unambiguous staining patterns.

Laser Capture Microdissection.

Using p16 staining we identified cases from surgical excisions that were strongly positive (>90% immunoreactivity for p16 with minimal criteria=>70%) or completely negative. Of the 109 cases stained, 53 had sufficient primary tumor tissue available for laser capture, performed on the ArcturusXT system (Invitrogen). For each case, a minimum of 8000 cancer cells were dissected and caps stored at −80 C. RNA purification utilized the miRNeasy FFPE kit (Qiagen). All samples were assessed with a Nanodrop spectrophotometer and Agilent Bioanalyzer 2100. Additionally, a limited number of samples were run on a miRNA QC PCR array as a means of positive control to assess stability of the small RNA fraction (Qiagen).

Real Time PCR Based miRNA Profiling.

Qiagen's miRnome global qPCR array, which includes assays for 1008 individual microRNA species, was utilized for profiling. As described below, each array consisted of three 384-well plates, such that each patient's sample was run on three separate plates. The 1008 sequences profiled represent all annotated miRNA sequences in the human miRNA genome (miRBase release 16). cDNA was synthesized with the miScript II RT kit and qPCR was performed using the Roche LightCycler480. Each assay was begun with hot start activation and included 45 cycles of amplification with melt curve analysis step for determining specificity of each probe (by calculating Tm). Absolute quantitation was used to generate threshold values (Cp) using the second derivative maximum algorithm unique to the LC480 system (auto baseline). Statistical analysis was performed using the geNorm algorithm. To improve the threshold of detection, pre-amplification was performed on cDNA synthesized with the miScript II RT kit using the miScript PreAMP PCR kit and miScript PreAMP Primer mix (Qiagen). Amplified cDNA was added to the miScript SYBR mix, water and MiScript universal primer and dispensed to miRNome plates. A test miRNome array was first utilized to determine if the dilution for real time PCR resulted in high percentage call rates. Pre-amplified samples including p16+(n=15) and p16− (n=9) samples were loaded on the miRnome plates (15 ng cDNA/sample). Subsequent analysis was performed as described above using Ct values≦30 as a cutoff for inclusion.

PPPE-Based miRNA Cohort Bioinformatics.

Raw data from the RT-PCR arrays were subjected to extensive quality control analyses based on specialized internal controls on the arrays including positive PCR controls, which test the efficiency of the polymerase chain reaction itself, and reverse transcription controls to detect any impurities that inhibited the RT phase of the procedure. We also calculated mean, standard deviation, and coefficient of variation and compared them to values published on FFPE cancer samples for these arrays. Philippidou et al, Cancer Res., 70(10):4163-73 (2010). Any sample that failed to fall within the acceptable range of metrics as defined by Qiagen was excluded from the analysis; one of our 24 samples failed this step. Next, miRNAs with Ct values of >30 (or 0) for pre-amplified samples were considered ‘not reliably detected’ and excluded from analysis by replacing that Ct with ‘NA’ to indicate missing. Determining reference genes for normalization was carried out on a plate-by-plate basis according to the geNorm algorithm, utilizing the R/Bioconductor package SLqPCR. Peltier, H J, Latham, G J, RNA, 14(5):844-52 (2008). To test which miRNAs were differentially expressed based on HPV status, we used the R/Bioconductor limma package. To focus on widely expressed miRNAs and to increase power, non-specific filtering of miRNAs was carried out as follows: only those miRNAs which were detected in >90% of samples were carried forward for subsequent analysis. Moderated t-statistics were applied to each miRNA using an Empirical Bayes approach, in which the standard errors are shrunk towards a common value. The comparison of interest was HPV+ versus HPV−, expressed in terms of fold change (HPV+/HPV−). The comparison was made after adjusting for smoking, smoking and HPV interaction, and age to account for potential confounding effects. In the event of a significant interaction between smoking and HPV for a specific miRNA, the interpretation of the effect of either smoking or HPV in isolation should be made with caution.

Validation of Oncogenic miRNA Panel Using Patient Cohorts from the Cancer Genome Atlas (TCGA).

Patient consent/enrollment and utilization of data were conducted in accordance with TCGA Human Subjects Protection and Data Access Policies. Clinical data were first downloaded for HNSCC patients (458 cases), HPV ISH or HPV p16 testing was assessed, and then patients with definitive status (positive or negative) were selected resulting in 66 cases. Of these, 11 cases were positive and 11 were definitively HPV− according to p16 and/or HPV− ISH. Annotated RNA-Seq data were then downloaded from the TCGA Data Portal in September and October 2013. Because the comparison of interest was HPV+ to HPV− cancer, individual normalized expression values for a particular miRNA were compared between groups of HPV+ patients (n=11) and HPV− patients (n=11) by using student's t-test and the results sorted according to the level of significance. For miRNAseq, the relative abundance of a particular microRNA is represented by the absolute number of sequence reads.

Validation by miRNA-In Situ Hybridization (miRNA-ISH) in HNSCC Tissue Microarrays.

HNSCC tissue microarrays (TMA) were assembled from cases available in the Department of Pathology & Immunology, Washington University School of Medicine, using tissues obtained with approval of the Human Research Protection Office. The TMA included 357 cases of HNSCC with two tumor tissue cores per case Immunohistochemistry was performed for p16 on a full FFPE section on a Ventana Benchmark automated immunostainer (Ventana Medical Systems, Inc., Tucson Ariz.) according to standard protocols with a known p16-expressing SCC case and normal tonsil as positive and negative controls, respectively. Antigen retrieval utilized the Ventana CC1, EDTA-Tris, pH 8.0 solution. Staining was read by one study pathologist (JSL), and all positive cases demonstrated both nuclear and cytoplasmic staining. Staining was graded in a quartile manner as follows: 0=no staining, 1+=1 to 25% staining; 2+=26 to 50%; 3+=51 to 75%, and 4+=76 to 100%. Cases were then classified as positive (any convincing expression) versus negative, and then separately as strong positive staining (3 or 4+) versus negative or weak staining (0, 1+, or 2+). More than 90% of cases were either strongly and diffusely positive or completely negative. The TMAs were then processed for RNA-ISH for HPV or miRNA-ISH for miR-9. In situ hybridization for high risk HPV E6/E7 RNA was performed using the RNAscope™ HPV kit (Advanced Cell Diagnostics, Inc., Hayward, Calif.) according to the manufacturer's instructions and classified by the study pathologist (JSL) as either positive or negative. Positive cases had granular cytoplasmic and/or nuclear brown staining that was above the signal on the negative control slide. In situ hybridization for miR-9 was performed at the Center for RNA Interference and Non-Coding RNAs at MD Anderson Cancer Center (MDACC, Houston, Tex.). Double digoxigenin (DIG) labeled (5′ and 3′) miRCURY LNA™ probes were obtained from Exiqon (Denmark). For visualization, the digoxigenins are detected with a polyclonal anti-DIG antibody and alkaline phosphatase conjugated secondary antibody using 5-bromo-4-chloro-3-indolyl-phosphate/nitro blue tetrazolium (BCIP/NBT). In these experiments, the full-length mature microRNA sequence for miR-9 was used for specific LNA probes: TCATACAGCTAGATAACCAAAGA (SEQ ID NO: 27). All tumor tissues stained for miR-9 were counterstained with nuclear fast red. One entire TMA was stained with LNA U6 snRNA probe as positive controls without counterstain. Each TMA contained normal reference tissues that served for orientation and as negative controls (liver, thyroid, or small bowel). All resulting slides were assessed (by DLM and/or JSL) in a blinded fashion as to HPV status, H&E morphology, and all other clinical-pathologic parameters. The stained slides were read and scored in a systematic fashion according to the following 3 parameters: (1) proportion of tumors cells with identifiable ISH signal (0<10%, 1=10-25%, 2=25-50%, 3=50-75%, 4=>75%); (2) strength of ISH signal (weak, weak/moderate, moderate, strong); (3) staining pattern (punctate, diffuse, or punctate+diffuse). The staining results were tabulated then unblinded and combined with data regarding HPV status and additional clinical data. The subsequent logistic regression analysis was performed separately for each binary outcome (HPV status measured by p16 and ISH) with the following for predictors: staining proportion, strength of ISH signal, and staining pattern. Model selection criteria were used to guide the selection of the final variables included in the model. Internal cross-validation was reported to indicate the performance of the model.

Results

Characterization of Oropharyngeal Tissues.

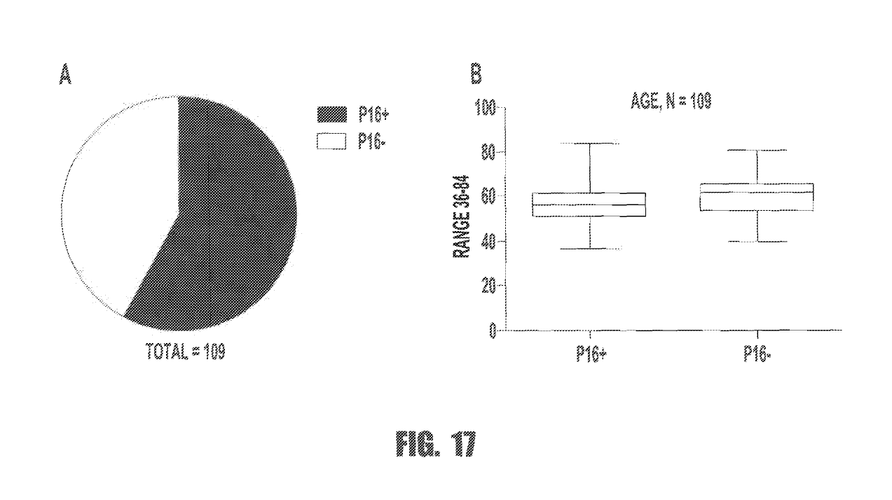

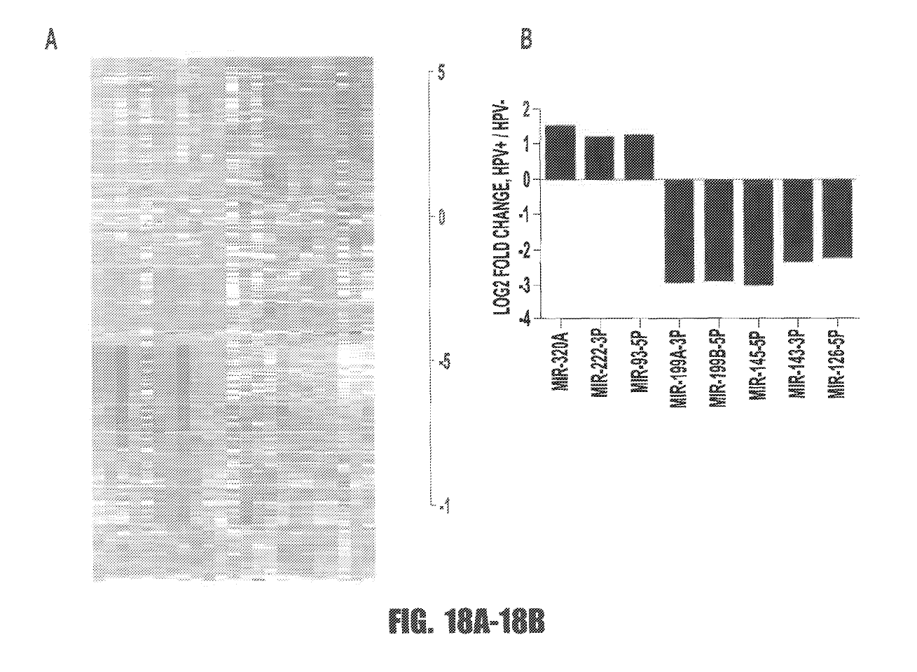

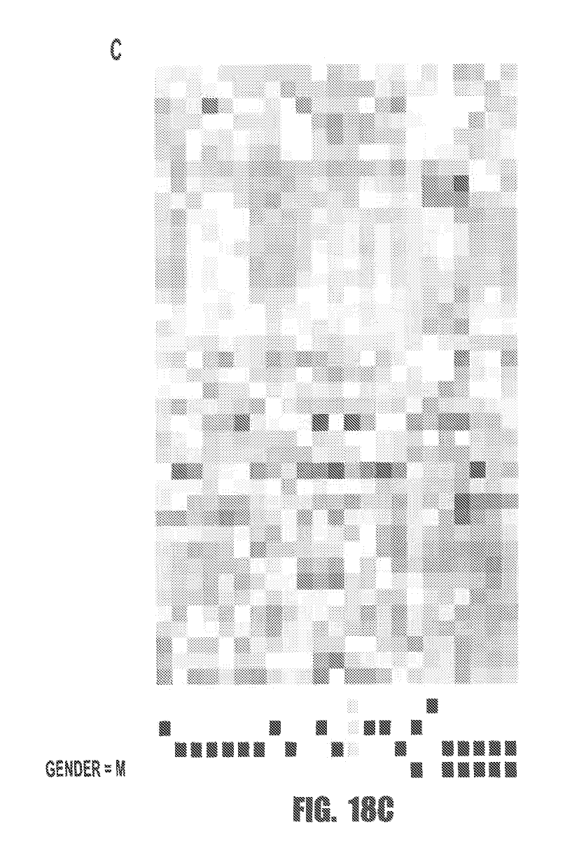

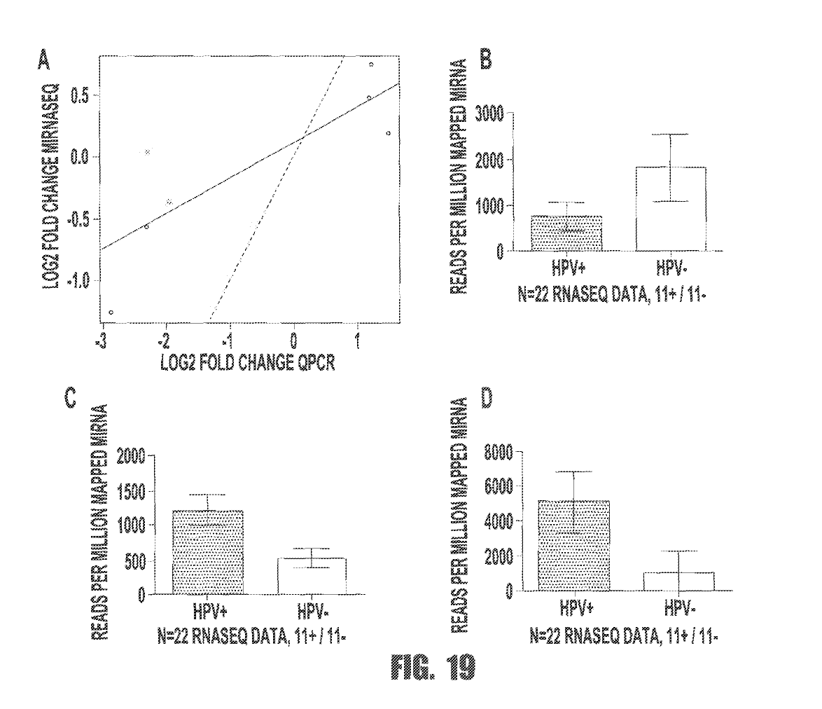

As national and international rates of HPV driven OPSCC have been called epidemic in scale, we therefore asked whether patients previously diagnosed with OPSCC at the University of Missouri reflect concordant epidemiological proportions. Of the cases (n=109) stained for p16 expression, 58% were positive (FIG. 17A). Multi-institutional US studies estimate 65-70% of OPSCC to be caused by HPV. Chaturvedi et al., J Clin Oncol, 29: 4294-301 (2011). The average ages in the cohorts under study were 56.49 and 61.00 years of age for p16+ and p16− respectively (FIG. 17B). HPV/OPSCC has a male predominance and no consistent association with smoking. Disease staging also has been associated with differences between HPV+ and HPV− OPSCC, with HPV+ disease more commonly associated with stage IV disease and frequent lymph node metastases at initial presentation. Overall, we interpret these clinical data as positive signal that our p16+ and p16− cohorts reflect true disease trends.

HPV+ and HPV− Tumors have Distinct MiRNA Profiles.

To identify a distinct miRNA signature that can differentiate HPV+ from HPV− OPSCC, we performed PCR-based miRNA profiling using a minimum of ten 10 μm sections from each of 24 cases. Following preamplification, improved signal detection is evident. One case was excluded based on quality control measures. Prior to non-specific filtering there were 511 miRNAs; afterward 276 remained and were used for modeling. Results from our linear model showed that three individual miRNA sequences were significantly up-regulated in HPV+ patients: miR-320a, miR-222-3p, and miR-93-5p. The most statistically significant downregulated miRNAs included 6 sequences, representing 4 unique mature miRNAs: miR-199a-3p//-199b-3p, miR-143, 145, and mir-126a (FIG. 18A,B and Table 6). The top 10 miRNAs that were most impacted by age or smoking status do not show fold changes of the magnitude we found associated with HPV status. Three miRNAs that showed a significant HPV effect also had a significant Smoking×HPV effect (miR-320a, miR-126, and miR-143; results not shown). The full expression matrix (511 miRNAs, 23 samples) was log₂ transformed and clustered using unsupervised hierarchical clustering based on average agglomeration with |1−rho| as the distance measure. The results are presented as a heat map, with dendrograms indicating the clustering of patient samples (columns) and miRNAs (rows) (FIG. 18A). Additional unsupervised hierarchical clustering was performed using a list of 43 miRNAs implicated by our own dataset as well as those from the literature (FIG. 18C). These analyses resulted in data that strongly supports the hypothesis that HPV+ oropharyngeal tumors display distinct miRNA profiles and that groups of miRNAs can be associated with an oncogenic HPV infection.

TABLE 6
Oncogenic miRNA profile from FFPE HPV+ vs HPV−
OPSCC cases. Table depicts fold change and log₂FC
for the 9 most statistically significant miRNA
sequences, representing 7 distinct miRNAs.

| | Fold Change | logFC | p-value |
|---|---|---|---|
| hsa-miR-320a | 2.83 | 1.50 | 2.02E−03 |
| hsa-miR-143-3p | 0.21 | −2.28 | 2.50E−03 |
| hsa-miR-222-3p | 2.31 | 1.21 | 4.58E−03 |
| hsa-miR-93-5p | 2.36 | 1.24 | 5.41E−03 |
| hsa-miR-199a-3p// | 0.14 | −2.87 | 5.32E−03 |
| hsa-miR-199b-3p | | | |
| hsa-miR-126-5p | 0.23 | −2.14 | 5.48E−03 |
| hsa-miR-145-5p | 0.20 | −2.30 | 5.91E−03 |
| hsa-miR-126-3p | 0.26 | −1.94 | 6.94E−03 |
| hsa-miR-199b-5p | 0.14 | −2.85 | 8.80E−03 |

Correlation of miRNA Expression with HPV Expression from TCGA Database Validates PCR-Based HPV− Associated miRNA Profile.

To further assess changes in the miRNA expression profile associated with HPV+OPSCC, we utilized publically available clinical and miRNA-seq data from The Cancer Genome Atlas (TCGA) and identified 11 primary OPSCC cases (tonsil or BOT) positive for either p16 IHC or HPV ISH that were paired with 11 HPVcases, which we designated as TCGA Cohort 1. In TCGA Cohort 1, we found 84 miRNAs to be significant (p<0.05 and mean RPKM>10 in both groups). Using the average RPKM for each group as general estimations of expression level, fold-changes were calculated (+/−) and cutoffs of log 2 fold changes of +/−1.0 were used, yielding a list of 36 miRNAs.

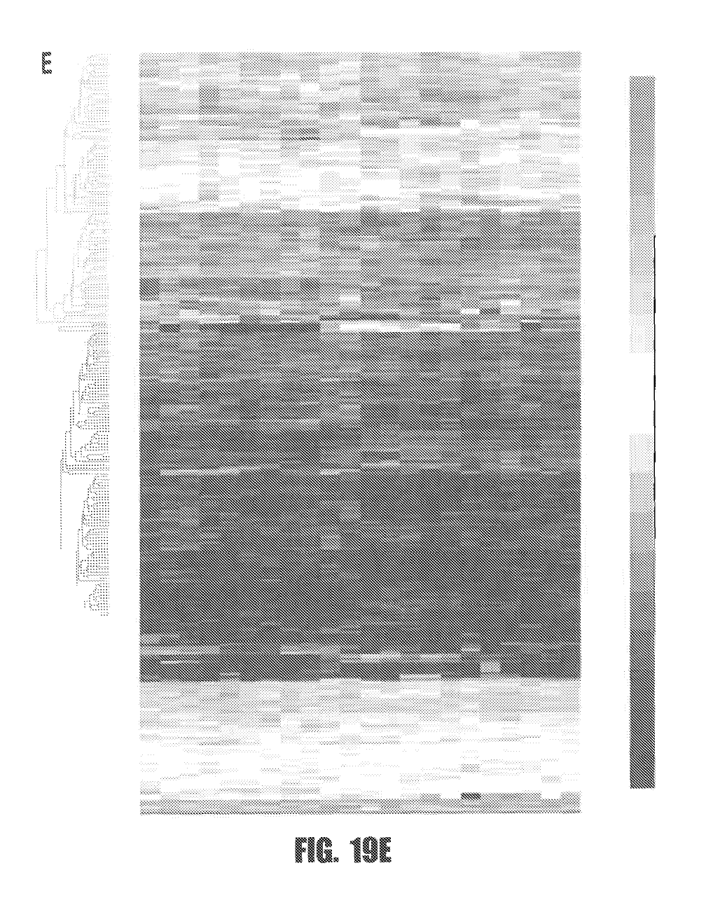

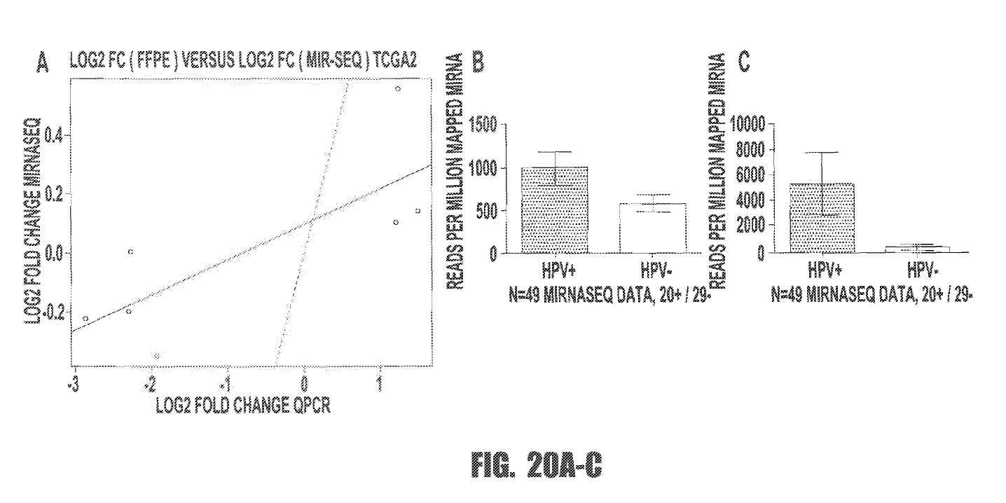

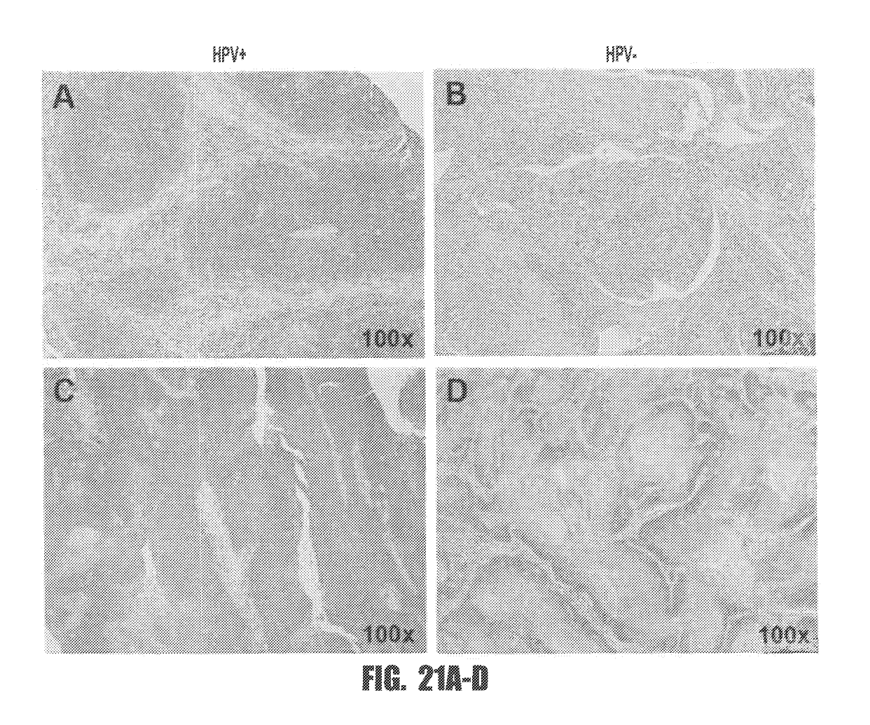

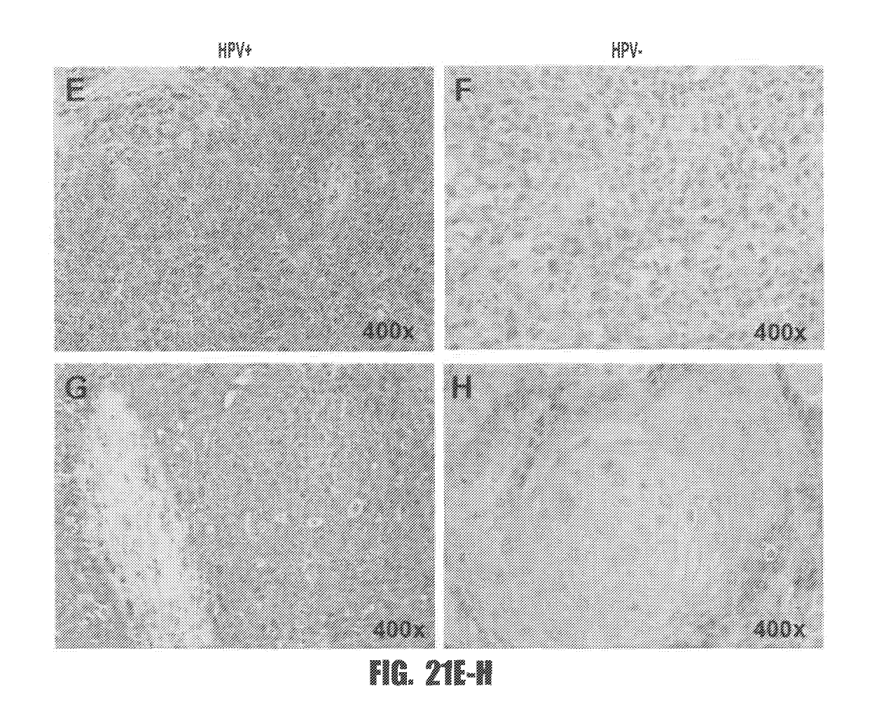

The results from TCGA Cohort 1 were compared to the panel of miRNAs identified from our PCR profiling of PIPE tissue. The fold changes of these 7 miRNAs between HPV+ and HPVpatients are concordant between the two datasets based on both Spearman's rank correlation (rho=0.85, p=0.02) and Pearson's correlation (r=0.83, p=0.02; 95% CI, 0.21-0.97) (FIG. 19A). Amongst the most statistically significant miRNAs from TCGA Cohort 1, 16 of which are shown in Table 7, miR-199-1, miR-106b, and miR-9 were highly correlated to patients who are HPV+ irrespective of other covariates such as age or smoking status (FIGS. 19B, C, D). Interestingly, miR-9 was upregulated 1.84 fold (p=0.14) in our FFPE qPCR cohort and it has been identified by two independent published studies as an HPV− associated miRNA in OPSCC. Therefore, the upregulation of both miR-9-1 and miR-9-2 in TCGA Cohort 1 miRNAseq data served as strong validation that this is an HPV-associated miRNA in OPSCC. Unsupervised clustering performed on the full miRNAseq data indicated that, in this dataset, HPV+ disease is associated with distinct miRNA profiles (FIG. 19E). Notably, a group of miRNAs that are highly expressed in both HPV+ and HPV− disease, including miR-21, miR-203, and miR-22 clustered at the bottom of this heat map, implicating these miRNAs as potentially important in squamous differentiation of HNSCC regardless of HPV status.

TABLE 7
TCGA Cohort 1 and Cohort 2 analysis. Table depicts fold change and log₂FC
for the most statistically significant miRNA sequences from each cohort.
RPKM = reads per kilobase of transcript per million reads mapped.

| Data | | | | Fold | | |
|---|---|---|---|---|---|---|
| Source | microRNA | HPV+ | HPV− | Change | logFC | p-value |
| TCGA | hsa-mir- | 1225 | 542 | 2.26 | 1.18 | 1.7E−05 |
| COHORT 1 | 106b | | | | | |
| | hsa-mir- | 54501 | 20645 | 2.64 | 1.40 | 4.8E−05 |
| | 148a | | | | | |
| | hsa-mir-625 | 306 | 111 | 2.76 | 1.46 | 8.8E−05 |
| | hsa-mir-335 | 113 | 41 | 2.71 | 1.44 | 5.4E−04 |
| | hsa-mir-9-1 | 5078 | 1054 | 4.81 | 2.27 | 5.4E−04 |
| | hsa-mir-9-2 | 5091 | 1055 | 4.82 | 2.27 | 5.8E−04 |
| | hsa-mir-214 | 13 | 46 | 0.30 | −1.75 | 8.9E−04 |
| | hsa-mir-337 | 10 | 36 | 0.29 | −1.79 | 1.3E−03 |
| | hsa-mir- | 24 | 7 | 3.49 | 1.80 | 1.7E−03 |
| | 378c | | | | | |
| | hsa-mir-29c | 4456 | 1204 | 3.70 | 1.89 | 2.5E−03 |
| | hsa-mir-598 | 38 | 14 | 2.68 | 1.42 | 3.3E−03 |
| | hsa-mir-107 | 133 | 52 | 2.55 | 1.35 | 5.2E−03 |
| | hsa-mir-150 | 2796 | 746 | 3.75 | 1.91 | 5.6E−03 |
| | hsa-mir- | 55 | 18 | 3.05 | 1.61 | 6.6E−03 |
| | 106a | | | | | |
| | hsa-mir-378 | 2154 | 742 | 2.90 | 1.54 | 6.7E−03 |
| | hsa-mir-20b | 152 | 9 | 16.61 | 4.05 | 7.4E−03 |
| TCGA | hsa-mir-20b | 47 | 7 | 6.79 | 2.76 | 2.6E−04 |
| COHORT 2 | | | | | | |
| | hsa-mir-9-2 | 5287 | 325 | 16.26 | 4.02 | 5.4E−04 |
| | hsa-mir-9-1 | 5282 | 326 | 16.18 | 4.02 | 5.4E−04 |
| | hsa-mir- | 990 | 587 | 1.69 | 0.75 | 5.4E−04 |
| | 106b | | | | | |
| | hsa-mir-574 | 44 | 69 | 0.63 | −0.66 | 6.8E−04 |
| | hsa-mir- | 101 | 256 | 0.40 | −1.34 | 9.0E−04 |
| | 193b | | | | | |
| | hsa-mir-363 | 30 | 7 | 4.38 | 2.13 | 9.3E−04 |
| | hsa-mir-16-2 | 23 | 11 | 1.99 | 0.99 | 1.7E−03 |
| | hsa-mir-15b | 521 | 269 | 1.93 | 0.95 | 1.7E−03 |
| | hsa-mir-25 | 11787 | 7377 | 1.60 | 0.68 | 1.8E−03 |

Further Validation Studies Using TCGA and miRNA-ISH.

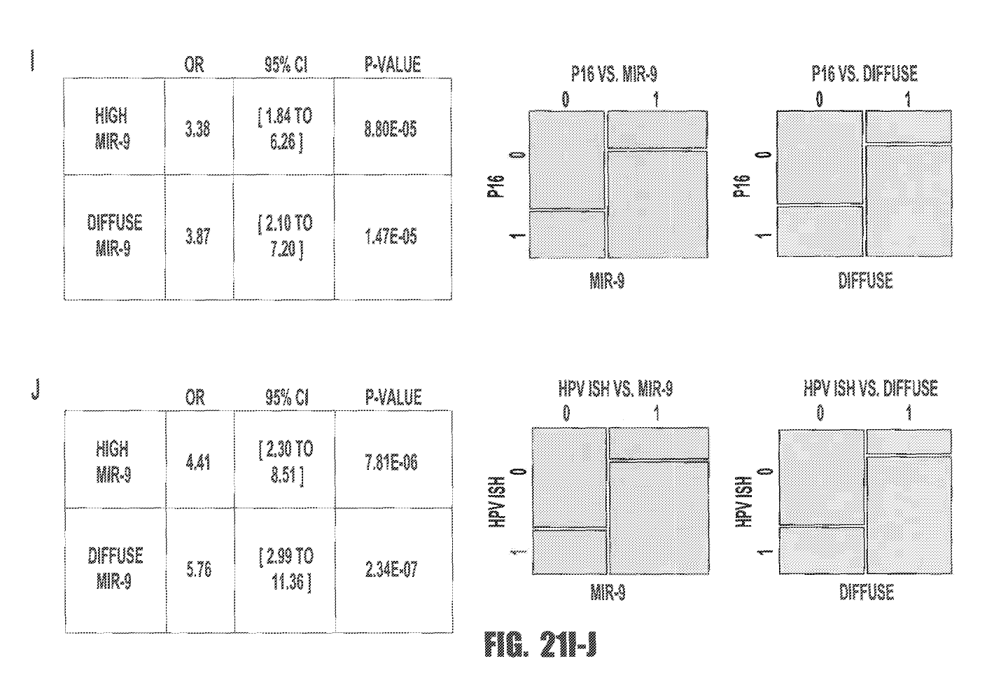

Based on a recent analysis of mRNAseq data from TCGA across 3,775 malignancies for the presence of viral sequences, a second expanded cohort of patients from TCGA with HPV-associated HNSCC was identified comprised of 20 cases that lacked available clinical data regarding p16 or HPV ISH, and were therefore not identified in our original analyses. Khoury et al., J Virol., 87(16): 8916-26 (2013). However, these 20 cases were shown to express various viral transcripts (18 cases expressed transcripts from HPV16 and 2 from HPV33) and have HPV DNA integrated into the host genome, were thus treated as HPV+, and were compared to an additional set of 29 HPV− cases (identified based on available p16 and/or HPV ISH data), herein designated TCGA Cohort 2. After extracting miRNAs that were expressed at suitable levels in both cohorts (>10 mean RPKM), a list of 43 miRNAs was generated, the top 10 of which are shown in Table 7. The fold changes of our 7 miRNA panel from FFPE studies show concordance between the two datasets based on Spearmans rank correlation (rho=0.75, p=0.06) and Pearson's correlation (r=0.78, p=0.03) (FIG. 20A). MiR-9 and miR-106b were again amongst the most statistically significant miRNAs. Both miR-9-1 and miR-9-2 are expressed at reads RPKM of ˜5000 in patients whose tumors express HPV transcripts. This is in stark contrast to patients whose tumors are negative for p16 IHC and/or HPV ISH, where on average miR-9 levels are ˜16-fold lower. The tissue level of miR-9 expression was also assessed via in situ hybridization on HNSCC tissue microarrays containing 357 cases including 270 OPSCC cases with known p16 status and 226 cases with known HPV RNA ISH results. Representative positive and negative punctate and diffuse staining is shown in FIG. 21A-H. The odds that high-tumor miR-9 expression occurs in the setting of p16+ disease are more than 3 times greater (OR=3.38, p<0.001; 95% CI 1.84-6.26) than the odds of low-tumor miR-9 expression; similarly, the odds of diffuse miR-9 ISH are nearly four times greater (OR=3.87, p<0.001; 95% CI 2.10-7.20) than the odds of non-diffuse miR-9 ISH (FIG. 21I). Interestingly, when the odds ratio calculations are based upon HPV mRNA in-situ hybridization, the ORs increase (OR=4.41, p<0.001, 95% CI 2.30-8.51) and (OR=5.76, p<0.001, 95% CI 2.99-11.36) for both miR-9 positivity and a diffuse cytoplasmic/nuclear pattern for miR-9 ISH signal (FIG. 21J). As has been shown in our own analyses of FFPE samples and analysis of TCGA Cohort 1 (p16/ISH confirmed cases), HPV-associated tumors are characterized by upregulation of miRNAs belonging to the miR-106b˜25 cluster (miR-106b, miR-93, miR-25). Our validation studies using TCGA Cohort 2 strongly support upregulation of members of this cluster in association to HPV status. Further, the expression levels of the closely related miR-106a˜363 cluster (miR-20b and miR-363) are tightly correlated to HPV status, although the expression levels of these miRNAs are low, making it difficult to interpret the potential biological significance (Table 7). It should be noted that others have reported upregulation of miR-20b as well as miR-363 based on qPCR profiling and microarray analyses of FFPE samples and cell lines, respectively. Wald et al., Head Neck, 33(4):504-12 (2011); Hui et al., Clin Cancer Res, 19: 2154-2162 (2013).

Discussion

HPV− Associated miRNA Profiling. The incidence rates for HPV+OPSCC have increased dramatically from 1988-2004, from 0.8 to 2.6 per 100,000, an increase of 225%. In striking contrast, HPV− negative cancers have declined 50%. These trends are also apparent internationally. Thus, it is of great interest to identify specific upregulated miRNAs characteristic of an oncogenic HPV infection of the head and neck, as these miRNAs may represent novel diagnostic/prognostic biomarkers and may provide a more detailed understanding of the molecular pathogenesis of the disease. We have provided miRNA profiles of three independent cohorts of patients (n=94) with SCC of the aerodigstive tract performed with PCR arrays and next generation deep sequencing. Comparative analysis of these cohorts strongly supports an HPV-associated upregulation of miR-9 and members of the miR-106b˜25 cluster and downregulation of miR-199-1.

The fundamental mechanism associated with HPV+ disease is a disruption of cellular differentiation induced by the virus, wherein the cell acquires resistance to growth inhibition, immune evasion, subversion of apoptosis, genomic instability, and ultimately dysregulated proliferation such that viral DNA can replicate in synchrony with chromosomal DNA. Thus, HPV may causally modulate miRNAs that then act as central nodes in affecting numerous genes important in progression and metastatic spread, resulting in divergent gene regulation. In contrast to cervical cancer, viral integration is not necessary for initiation of oncogenesis in OPSCC and episomal HPV DNA appears to be a frequent occurrence in tonsillar carcinomas. Syrjanen S., J Clin Pathol., 57(5):449-55 (2004). However, similar to cervical cancer, transcriptionally-active HPV is linked to the differentiation state of the host cell and in some circumstances leads to aberrant cell proliferation and genomic instability that is due in part to mitotic spindle defects that result in cytogenetic abnormalities. Duensing et al., Environ Mol Mutagen., 50(8): 741-7 (2009). Alternatively, miRNAs could be suppressed due to aberrant DNA methylation or chromosomal disruption. Johannsen E, Lambert P F., Virology, 445(1-2):205-12 (2013). As an individual miRNA can affect hundreds to thousands of genes, many of these often lie in the same biological pathway. Lal et al., PLoS Genetics 7(11): e1002363 (2011). Thus, specific cellular pathways important for immune surveillance, mitogenic signaling, or metabolism, that are integral for HPV infection, could also lead to typified miRNA expression.

Several studies have examined miRNA expression in the context of HPV and head and neck cancer, with heretofore a lack of consensus between reports. Lajer et al., Br J Cancer, 104(5): 830-40 (2011); Gao et al., Cancer, 119: 72-80 (2013). This may be due, in part, to the biological redundancy in miRNA function, the inherent genomic instability characteristic of HPV+ tumors, and the use of differing profiling methodologies and sample sizes. It is interesting to note that miR-20b shares significant sequence homology with miR-106b and miR-93, these miRNAs share the same seed sequence, and are closely related to miR-363. It has been reported that members of the miR-106b˜25 cluster are upregulated independent of HPV status relative to normal tonsillar epithelium (n=88 vs n=7). This is in contrast to the current study showing upregulation of members of the miR-106b˜25 cluster in HPV+OPSCC relative to HPV− as determined in the PIPE PCR-based miRNA profile, TCGA Cohort 1, and TCGA Cohort 2. Recent in vitro experiments that sought to identify miRNAs altered as a function of HPV transfection utilized deep sequencing on organotypic raft cultures derived from normal or HPV− 31-transfected human foreskin keratinocytes isolated from the same donor, demonstrating 2-3 fold upregulation of miR-106b and miR-25 in the HPV− transfected cultures relative to normal. Gunasekharan V, Laimins L A., J Virol. 87(10):6037-43 (2013). In the context of the current human tumor data, this study strongly supports that these miRNAs are altered as a function of HPV oncoproteins.

The TCGA deep sequencing data sets analyzed here add another dimension to available data on the expression of miRNAs shown to be statistically significant between HPV+ and HPV− disease, as normalized read counts provide information on the relative abundance of a specific miRNA. Thus, it is interesting to note that miR-363, which has been shown to be upregulated via microarray analysis in HPV+ disease by two independent studies, shows relatively low expression overall (<50 RPM in HPV+ and HPV− cohorts), yet the fold change for this miRNA is quite high. Similar in this regard is miR-20b, part of the same transcriptional unit and also previously reported as upregulated in HPV+ disease. In the current study, we observed upregulation of miR-20b (16- and 7-fold in TCGA Cohorts 1 and 2, respectively); however, it is also expressed at relatively low levels. In contrast, miR-106b and members of the miR-106b˜25 cluster are expressed at levels ˜30-fold higher. As the biological significance of these fold differences are unknown, the relative importance of the two clusters in HPV+OPSCC should be addressed experimentally, as it is unclear whether expression of these miRNAs at very low levels may nevertheless regulate important biological functions.

Upregulated miRNAs.

“MiRNA families” are determined based on common seed sequence and are predicted to target overlapping sets of genes. miR-106b˜25 and miR-106a˜363 are genomic paralogs of the miR-17˜92 cluster, one of the best characterized groups of miRNAs in human cancer, with oncogenic function in lymphoma, multiple myeloma, medulloblastoma, lung, and colorectal cancer. Ventura et al., Cell, 132(5):875-86 (2008). Interestingly, miR-106b, miR-93, and miR-20b are members of the miR-17 family; as such their seed sequence is identical to that of miR-17, 20a, 20b, 106a, and 106b and they are predicted to have redundant function. Concepcion et al., Cancer J., 18(3):262-7 (2012). However, the biological implications of intra-cluster redundancy are not entirely understood, as clustering of microRNAs with similar seed sequences is highly conserved, which suggests that members of the same cluster with identical seed sequences may have functional importance.

Members of the miR-106b˜25 and/or miR-106a˜363 cluster appear sensitive for differentiating HPV+ from HPV− HNSCC, however the mechanistic basis for upregulation is not entirely clear. HPV+HNSCC and cervical cancers are characterized by upregulated expression of distinct and larger subsets of cell cycle and DNA replication genes and transcription of miR-106b˜25 is concurrent with the protein coding gene MCM7. Indeed, MCM7 is a known RB/E2F target gene, and E2F family transcription factors may be paramount in mediating miR-106b˜25 and MCM7 transcription. The MCM7 promoter has RB/E2F binding sites and mRNA expression correlates with miR-106b expression. This is provocative since MCM7 has been proposed as a protein biomarker that distinguishes between HPV+ and HPV− HNSCC. Thus, the miR-106b˜25 cluster members may be sensitive for differentiating HPV+ from HPV− HNSCC as relatively increased expression of this cluster is highly characteristic of HPV+ tumors.

There is also considerable evidence that miR-106b˜25 and its paralogs can act as bona fide oncogenes (oncomirs) with defined roles in overcoming TGF-beta mediated growth suppression, enhancing TGF-beta signaling, cell cycle promotion, and increased cell survival. Petrocca et al., Cancer Cell., 13(3): 272-86 (2008). Repression of MCM7 together with miR-106b˜25 expression is associated with induction of p21 and PTEN in breast and prostate cancer. In neuroblastoma, miR-17˜92 expression induced potent inhibition of key TGF-β signaling effectors and direct inhibition of TGF-β responsive genes. Increased expression of miR-106b˜25 and miR-17˜92 in retinoblastoma was also reported, leading to the hypothesis that in the context of genetic Rb loss, high miR-17˜92 expression may circumvent the need for large numbers of genetic hits required for tumorigenesis. These results highlight the importance of context in miRNA oncogenic function and suggest the hypothesis that the current finding of miR-93, miR-106b, miR-20b, and miR-363 overexpression in OPSCC tumors supports the oncogenic activity instigated by the main viral oncoproteins, E6, E7, and E5.

In addition to the miR106b˜25 cluster, strong upregulation of miR-9 is also observed in HPV+OPSCC. miR-9 has been linked to metastatic potential via modulation of E-cadherin, acting to prime breast cancer cells for an epithelial-mesenchymal transition (EMT) and stimulate angiognesis. Because lymph node metastasis is extremely common in patients with HPV+OPSCC, and increased miR-9 levels in HPV+ relative to HPV− OPSCC were observed in the current study and others (Gao et al., Cancer, 119: 72-80 (2013)), we asked if there was a relationship between miR-9 and Ecadherin expression in OPSCC using qRT-PCR and western blotting of OPSCC cell lines of known HPV; however no relationship between miR-9 and Ecadherin was observed. Expression of E-cadherin is generally inversely correlated to prognosis in HNSCC, with early studies showing reduced E-cadherin as an independent prognostic marker for metastasis and local recurrence. A recent study (n=152) reported that the expression of E-cadherin is extensive in OPSCC with no correlation between E-cadherin and HPV, nodal or distant metastasis, histopathological type, or survival. Ukpo et al., Head Neck Pathol., 6(1): 38-47 (2012). A second analysis (n=102) suggested that HPV+/p16+ tumors from the oropharynx express high E-cadherin and β-catenin. Rampias et al., Ann Oncol., 24(8): 2124-31 (2013).

Together these results indicate that high miR-9 in HPV+OPSCC is not directly altering Ecadherin expression, suggesting that other functionally important miR-9 targets should be explored. Interestingly, miR-9 may modulate the microenvironment in HPV+OPSCC, as there is compelling evidence that miR-9 is packaged into microvesicles or may function as a tumor suppressor. Zhuang et al., EMBO J 31: 3513-3523 (2012). Physiological miR-9 expression may temper innate immune responses. In cancer, there is limited evidence that miR-9 may be involved in modulating immunoregulatory genes, including MHC Class I and interferon regulated genes. Data surrounding miR-9 and adaptive immune response is also lacking. Nonetheless, the biology associated with adaptive immune response in HPV+OPSCC is of particular interest and clinically relevant as these tumors are being considered for immune-checkpoint inhibitor therapy with anti-PD-1 or PD-L1 antibodies as well as adoptive cell transfer therapy. It has also been shown that the majority of transcriptionally active HPV+OPSCC tumors express PD-L1 (also called B7-H1), although the significance of this and its relevance to patient survival may be limited. Lyford-Pike et al., Cancer Res., 73(6): 1733-41 (2013). Thus, in light of strong upregulation of miR-9 in HPV+OPSCC, future studies will correlate miR-9 expression and the presence of immune infiltrates.

Downregulated miRNAs.

Downregulation of miR-143, miR-145, mir-199a-3p, miR-199b˜3p, miR-199b˜5p, and miR-126 has been observed in HPV+OPSCC and cervical disease. Lajer et al., Br J Cancer, 106:1526-1534 (2012). Similar results were obtained in an organotypic keratinocyte raft culture model, showing significant downregulation of miR-199a-5p and miR-145, suggesting a potential mechanistic link to early stages of the HPV life cycle. Our analyses on non-pre-amplified patient samples offered preliminary suggestions that miR-199 and miR-145 were also downregulated in patient samples (data not shown). After analyzing amplified samples, and calculating relative expression of miRNAs based on the geNorm algorithm, an extremely robust and informatics intensive method for analyzing q-RT-PCR data, our dataset confidently supports an HPVspecific downregulation of miR-145. The seed sequence of miR-145 is present in the E1 ORF of a number of papillomaviruses and E1 has been shown to be a bona fide miR-145 reactive element. Moreover, miR-145 plays a role in modulating the viral life cycle, with a differentiation-dependent reduction in levels of this miRNA in HPV transfected compared to control keratinocyte raft cultures that appears to be dependent on the function of viral E7 protein. Lentiviral forced expression of miR-145 plays a role in controlling genome amplification with a significant reduction of episomal viral DNA in undifferentiated cells. These in vitro data should be interpreted as having true biological significance, since other studies evaluating miRNA profiles in HNSCC and cervical cancer corroborate an HPV-associated down regulation of miR-145 and miR-143. Lajer et al., Br J Cancer, 104(5): 830-40 (2011). Taken together, these data indicate that HPV may downregulate miR-145 in order to allow for differentiation-specific genome amplification, and that this miRNA remains repressed in human tumors. In this context, we can understand miR-145 as a putative tumor suppressive miRNA in HPV disease.

### Conclusions

The overwhelming majority of HPV+HNSCC tumors arise deep within the crypts of the tonsils of the oropharynx. These tumors are often obscured from routine gross visualization in dental exams and, as such, most patients present with lymph node metastases. However, relative to patients with HPV− OPSCC, HPV+ status is positively correlated with a 2-3-fold increase in overall survival, indicating that the time is rapidly approaching whereupon HPV status will dictate therapy. The HPV-associated oncogenic miRNA panel identified herein may be incorporated into a multi-target diagnostic platform that can contribute to early detection and/or disease stratification to aid in differentiating oropharyngeal tumors with different prognoses and thus distinct management strategies. Furthermore, the oncogenic miRNA panel will facilitate mechanistic elucidation of molecular factors that contribute to OPSCC development, progression and response to therapy.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A probe for detecting oncogenic Human papillomavirus (HPV), comprising a polynucleotide that has at least 90% sequence identity to a polynucleotide complementary to at least one microRNA that has altered expression in response to oncogenic HPV infection.

2. The probe of claim 1, wherein expression of the microRNA is up-regulated in response to oncogenic HPV infection.

3. The probe of claim 1, wherein expression of the microRNA is down-regulated in response to oncogenic HPV infection.

4. The probe of claim 2, wherein the up-regulated microRNA is selected from the group consisting of SEQ ID No:1, SEQ ID No:2, SEQ ID No:3, SEQ ID No:4, SEQ ID No:5, SEQ ID No:6, SEQ ID No:7, SEQ ID No:8, and SEQ ID No:9.

5. The probe of claim 4, wherein the polynucleotide that that has at least 90% sequence identity to a polynucleotide complementary to the up-regulated microRNA is selected from the group consisting of SEQ ID No:14, SEQ ID No:15, SEQ ID No:16, SEQ ID No:17, SEQ ID No:18, SEQ ID No:19, SEQ ID No:20, SEQ ID No:21, and SEQ ID No:22.

6. The probe of claim 3, wherein the down-regulated microRNA is selected from the group consisting of SEQ ID No:10, SEQ ID No:11, SEQ ID No:12, and SEQ ID No:13.

7. The probe of claim 6, wherein the polynucleotide that that has at least 90% sequence identity to a polynucleotide complementary to the down-regulated microRNA is selected from the group consisting of SEQ ID No:23, SEQ ID No:24, SEQ ID No:25, and SEQ ID No:26.

8. A method for determining if a subject is infected by an oncogenic HPV, comprising:
obtaining a sample from the subject;
determining the level of a microRNA whose expression is altered in response to infection by an oncogenic HPV,
comparing the level of the microRNA to a control level, and
determining that the subject is infected by an oncogenic HPV if the level of the microRNA is altered relative to that of the control level.

9. The method of claim 8, wherein the microRNA is up-regulated in response to oncogenic HPV infection.

10. The method of claim 8, wherein the microRNA is down-regulated in response to oncogenic HPV infection.

11. The method of claim 9, wherein the up-regulated microRNA is selected from the group consisting of SEQ ID No:1, SEQ ID No:2, SEQ ID No:3, SEQ ID No:4, SEQ ID No:5, SEQ ID No:6, SEQ ID No:7, SEQ ID No:8, and SEQ ID No:9.

12. The method of claim 11, wherein a probe complementary to the up-regulated microRNA, is selected from the group consisting of SEQ ID No:14, SEQ ID No:15, SEQ ID No:16, SEQ ID No:17, SEQ ID No:18, SEQ ID No:19, SEQ ID No:20, SEQ ID No:21, and SEQ ID No:22.

13. The method of claim 10, wherein the down-regulated microRNA is selected from the group consisting of SEQ ID No:10, SEQ ID No:11, SEQ ID No:12, and SEQ ID No:13.

14. The method of claim 13, wherein a probe complementary to the down-regulated microRNA is selected from the group consisting of SEQ ID No:23, SEQ ID No:24, SEQ ID No:25, and SEQ ID No:26.

15. The method of claim 8, wherein the level of the microRNA is determined using an assay selected from the group consisting of RT-PCR, Fluorescence In Situ Hybridization and use of a microfluidic chip.

16. The method of claim 15, wherein the level of the microRNA is determined using an RT-PCR assay.

17. The method of claim 15, wherein the level of microRNA is determined using Fluorescence In Situ Hybridization using a probe selected from the group consisting of SEQ ID No:14, SEQ ID No:15, SEQ ID No:16, SEQ ID No:17, SEQ ID No:18, SEQ ID No:19, SEQ ID No:20, SEQ ID No:21, and SEQ ID No:22.

18. The method of claim 15, wherein the level of microRNA is determined using a microfluidic chip and a probe selected from the group consisting of SEQ ID No:14, SEQ ID No:15, SEQ ID No:16, SEQ ID No:17, SEQ ID No:18, SEQ ID No:19, SEQ ID No:20, SEQ ID No:21, and SEQ ID No:22.

19. The method of claim 8, wherein the sample is an oral rinse.

20. The method of claim 19, wherein the oral rinse has a volume of at least 10 mL.
